(19) United States
(12) Patent Application Publication
(10) Pub. No.: US 2002/0127561 A1
(43) Pub. Date: Sep. 12, 2002

(54) Assay for genetic polymorphisms using scattered light detectable labels

(76) Inventors: Gary Bee, Vista, CA (US); David E. Kohne, La Jolla, CA (US); Linda Korb, San Diego, CA (US); Todd Peterson, Coronado, CA (US); Juan Yguerabide, La Jolla, CA (US)

Correspondence Address: Wesley B. Ames, FOLEY & LARDNER, 23rd Floor, 402 West Broadway, San Diego, CA, 92101-3542, US

(21) Appl. No.: 09/880,732

(22) Filed: Jun. 12, 2001

Related U.S. Application Data
(60) Provisional application No. 60/210,988, filed on Jun. 12, 2000.

(51) Int. Cl. C12Q 1/68; C12P 19/34

(52) U.S. Cl. 435/6; 435/91.2

(57) ABSTRACT

Described are methods for determining the presence or absence of particular polymorphisms in CYP2D6 and other genes using scattered light detectable particles as detectable labels, and compositions useful in such methods.

### RELATED APPLICATIONS

This application claims the benefit of Bee et al., U.S. Provisional Application 60/210,988, entitled Assay for CYP2D6 Alleles, which is hereby incorporated by reference in its entirety, including drawings.

### BACKGROUND OF THE INVENTION

Colloidal gold particles have been used for a number of different applications, including in electron microscopy. However, it has also been found that gold particles, and other metallic particles can also serve as highly sensitive labels in bio-analytical assays and in the design, manufacture and quality control of small fluid volume instruments, devices, and processes by functioning as resonance light scattering (RLS) particles.

A number of different methods have been described for preparing metallic particles in the size range of about 1 nanometer to about 1 micrometer. For gold and silver particles, these methods have generally involved the addition of a reducing agent to a solution of metal ions, generating a population of gold or silver particles with a wide distribution of sizes. Typically, such particles have then been utilized as metals sols, or colloids, either directly, or with crude size fractionation to reduce the magnitude of size distribution.

Commercial preparations of gold particles have been provided in a variety of different sizes. Gold particles in the range of 5 nm to 20 nm have been provided for use in various bio-analytical test systems. Recently, smaller particles, below 5 nm and even sub-nm sizes have been used in histochemical applications. Colloidal gold particles in the sub-nm to 20 nm size range are usually prepared in a single step with a suitable reducing agent. (See, e.g., Colloidal Gold: Principles, Methods, and Applications, Vol. 1) Particles within these size ranges are typically used in electron microscopic methods or for assays where a result is produced that is visible to the unaided eye or with the use of photometric equipment. (See, e.g., product literature from colloidal gold manufacturers such as British Biosciences International)

The diameter of colloidal gold particles is dependent on a number of factors. The selection of an appropriate reducing reagent and its concentration in the reaction, the temperature, and the concentration of the water soluble gold salt are some of the important factors. Typically, a specific reducing agent is selected to prepare colloidal gold particles in a single step. That is, particles are nucleated and grown to a diameter predetermined by the nature of the reducing agent, its concentration in the reaction milieu, temperature, and concentration of gold salt.

The preparation of large (diameter greater than 20 nm) gold particles in a single step process usually produces populations of particles with broad size distributions.

### SUMMARY OF THE INVENTION

The present invention concerns a method for determining the presence of particular single nucleotide polymorphisms, or alleles, in genomic nucleic acid, preferably a pharmacogenetically relevant gene or genes in a DNA sample, for example, in a sample containing nucleic acid corresponding to CYP2D6, and provides convenient and sensitive detection of identified genetic polymorphisms. Such polymorphisms include, for example, deletions, insertions, and single nucleotide polymorphisms (SNPs).

The method utilizes a detection method based on the use of certain particles of specific composition, size, and shape and the detection and/or measurement of one or more of the particle's light scattering properties. The detection and/or measurement of the light-scattering properties of the particle is correlated to the presence, and/or amount, or absence of one or more analytes in a sample. The present invention is versatile and has utility in one form or another to detect and measure one or more target sequences in a sample. Such methods preferably utilize methods for analyte detection as described in Yguerabide at al., PCT/US97/06584, Yguerabide et al., PCT/US98/23160, Yguerabide et al, U.S. Pat. No. 6,214,560, and Yguerabide et al., U.S. application Ser. No. 08/953,713. Additional methods include those described in Swope, U.S. Pat. No. 5,350,697, Schutt, U.S. Pat. No. 4,979,821, and Stimpson, U.S. Pat. No. 5,843,651.

The invention features a method for detection of one or more target sequences, e.g., CYP2D6 target sequences, in a sample by binding those target sequences to at least one detectable light scattering particle, preferably with a size of 1-500 nm, generally smaller than the wavelength of the illumination light. This particle is illuminated with a light beam. Preferably the illumination is under conditions where the light scattered from the beam by the particle can be detected by the human eye with less than 500 times magnification. The light that is scattered from the particle is then detected under those conditions as a measure of the presence of those one or more target sequences. Applicant has determined, by simply ensuring appropriate illumination and ensuring maximal detection of specific scattered light, that an extremely sensitive method of detection results.

The method and associated apparatus are designed to maximize detection of only scattered light from the particles and thus is many times more sensitive than use of fluorophores. Such particles can be detected by using a low magnification microscope (magnifying at 2 to 500 times, e.g. 10 to 100 times) without the need for any electronic amplification of the signal. In addition, methods are provided in which no microscope or imaging system is necessary, but rather one or more of the light scattering properties are detected in a liquid or on solid-phase sample through which light is scattered. These scattered light properties can be used to determine the presence, absence or amount of analyte present in any particular sample. However, for some embodiments, electronic detection systems are advantageous and are used, e.g., for quantitative or semi-quantitative analyses, for particle counting, and for automated or semi-automated systems, or when computer-based analysis or further processing is desired.

The invention includes a number of different methods for preparation of probes, primers, and targets; labeling of probes and target, and attachment and detection of light scattering particle labels.

Thus, in a first aspect, the invention provides a method for determining the presence or absence of a target sequence e.g., a CYP2D6 target sequence in a sample of DNA containing nucleic acid corresponding to the gene. The method involves contacting the nucleic acid sample with a probe or probes under stringent binding conditions, and detecting the presence or absence of target sequence(s) bound with the probe or probes. In preferred embodiments, the probe (or probes) is bound with a scattered light detectable particle, and the detecting involves observing light scattered from said particle as an indication of the presence or absence of the target sequence or sequences. In some embodiments, the target molecule(s) is bound with a scattered light detectable particle.

In preferred embodiments, the method also involves amplifying a portion or portions of the nucleic acid corresponding to CYP2D6, e.g., using PCR, and contacting the amplified nucleic acid with the probe.

Also in preferred embodiments, the nucleic acid corresponding to CYP2D6 or other gene is immobilized on a solid surface. The nucleic acid can be immobilized using any of a variety of techniques, typically methods known in the art. These include, for example, binding to capture probes attached to a solid phase surface, direct adsorption of the nucleic acid to a membrane, filter, glass, or plastic, or attachment through a binding pair interaction other than nucleic acid sequence hybridization, for example, biotin/avidin or antigen/antibody, or any of a variety of other binding interactions known in the art. A number of different formats can be used, for example, microtiter plates (e.g., 96-well, or 256-well plates), glass slides, plastic slides, filters, and membranes. On slides, filters, membranes and the like, a single immobilization spot may be used, but preferably a plurality of spots are used, e.g., at least 5, 10, 20, 30, 40, 50, 80, 100, 200, 500, 1000, or even 5000, 10000, or more. In preferred embodiments, there are between 5 and 10, 10 and 20, 15 and 30, 20 and 40, 30 and 60, 50 and 100, 100 and 200, 200 and 400, 400 and 1000, 1000 and 5000, or 5000 and 10,000 spots. The specified number of spots can include control spots, or be exclusive of control spots.

Preferably a plate or slide or membrane or other solid phase formats includes control spots. Such control spots, can for example, include one or more of positive binding control, negative binding control, and amplification control, e.g., CYP2D7 amplification control, and CYP2D8 amplification control spots. A plurality of different spots preferably have different immobilized nucleic acid molecules corresponding to a particular gene, e.g., CYP2D6 nucleic acid molecules. Other nucleic acid molecules can also be immobilized on the same solid phase device.

While it can be useful to detect a single target sequence, in preferred embodiments, the method involves determining the presence or absence of a plurality of target sequences in nucleic acid corresponding to a particular gene, e.g., CYP2D6, using a plurality of probes. The probes bind to a plurality of different target sequences.

Preferably the method is used to identify the presence or absence of a plurality of different specific sequence polymorphisms or mutations. As indicated, these can be identified by various methods, particularly including allele specific nucleic acid probe hybridization and allele specific amplification or extension. Such allele specific hybridization is commonly arranged such that a nucleic acid probe will be perfectly complementary to a target sequence including a polymorphic site in at least one allele, but will have at least one mismatched nucleotide in at least one other allele. Typically, the probe is designed to possess a maximum kinetic or stability difference between the homologous complementary target and the corresponding polymorphic allele.

Similarly, an amplification oligonucleotide, such as a PCR primer can be arranged so that it will preferentially extend or amplify when there is complementary base pairing at the 3′ end, as compared to where there is not complementary base pairing at the 3′ end. As a result, there will be significantly more nucleic acid amplification product for the matched sequence, and the presence of that sequence can be identified, thereby identifying the presence or absence of particular sequence at that polymorphic site.

Preferably, the presence or absence of the plurality of target sequences identifies at least one allele of the particular gene, e.g., a CYP2D6 allele. Preferably, allelic forms of both copies of the gene are determined. Further, instead of merely determining the genotype, it is preferable to determine the sequence at a polymorphic site or sites for both copies of the gene. Those skilled in the art are familiar with determining a genotype for both copies of a gene (or for 3, 4, up to all copies of a gene when there are multiple functional copies of the gene). In identifying the sequences at a plurality of polymorphic sites, preferably the gene is identified as being one of a plurality of different alleles of the gene, e.g., CYP2D6 alleles. As described below, a large number of different allelic forms of the CYP2D6 gene are known, and it is useful to distinguish between them. Similarly, multiple allelic forms of other genes listed herein are known, and the present invention allows the various alleles to be conveniently distinguished.

In the context of this invention, the term “scattered light detectable particle”, “light scattering particle” and similar terms refer to particles that are of a size and composition such that light scattered from the particles on illumination with white light can be detected by human eye with no more than 500× magnification, preferably no more than 100× magnification. Generally such particles comprise a metal or metal-like substance, or combination of such substances in sufficient quantity to provide the required light scattering intensity. Also generally, such particles cross-sectional size of 1-500 nm, preferably 20-200 nm, more preferably 40-120 nm. Preferably such particles include gold and/or silver. Thus, particles referred to herein as RLS particles are light scattering particles.

In reference to nucleic acid sequences in the invention, the terms “amplifying” and “amplification” refers to an in vitro enzymatic nucleic acid synthesis providing an increase in the numbers of molecules having the sequence being amplified of at least two-fold, but usually at least 100 fold, or 1000-fold, or more. Increase in numbers less than 100-fold, will be referred to as “low level amplification”.

In the context of this invention, the term “allele” or “allelic form” refers to a form of a gene, e.g. CYP2D6 gene, containing a specified set of sequences at a particular set of polymorphic sites. For CYP2D6, for example, the presence of particular sets of sequences at such sites correlates with functional level of the gene product, e.g., CYP2D6 gene product. Unless expressly stated to the contrary, specification of an allele (allelic form) of a gene does not require specification of nucleotide sequence at all polymorphic sites, but neither the specification of sequence at a particular set of polymorphic sites. Such a set can include e.g., at least 1, 2, 4, 6, 8, 10, or more such polymorphic sites.

In connection with oligonucleotide-based assays, e.g., on arrays, the term “capture probe” refers to an oligonucleotide-containing molecule that hybridizes to a target molecule and allows the target molecule to be removed or otherwise separated from bulk sample. The capture probe nucleotide sequence may provide gene-specific and/or allele-specific binding.

Also in connection with oligonucleotide-based assays, the term “detection probe” refers to an oligonucleotide-containing molecule that hybridizes to a target molecule and provides detectability for the presence of the target molecule. The detection probe can be gene specific and/or allele-specific. It can also include a moiety or moieties that provide binding to light scattering particles, or can be bound to such a particle. In exemplary assays, a capture probe is used to bind a target molecule to a location on an array, and a detection probe is used to associate a detectable label with the immobilized target molecule.

In connection with solid phase arrays, the term “spot” refers to a defined location on the solid phase (e.g., a well, depression, or discrete location on a flat surface) that contains probe and/or target molecules. Typically, a probe and/or target molecule is bound to the solid phase in a spot. Spots in arrays are also commonly referred to as “features”.

As it is known that there are expressed but non-functional genes and non-expressed pseudo-genes with a high level of sequence similarity to CYP2D6, it is highly preferable to include control determinations to show that the sequence being detected or amplified is actually from CYP2D6 and not from one or more of the non-functional and/or pseudo-genes. One identified expressed but non-functional gene has been identified as CYP2D7, An identified pseudo-gene has been designated as CYP2D8. Thus, preferably a control or controls is included to demonstrate that one or both of these genes is not amplified. Similar controls are preferably included for other genes that have pseudogenes or expressed, non-functional related genes.

In methods involving amplification, the amplification can be carried out using various methods known in the art, specifically including the polymerase chain reaction (PCR).

In particular embodiments, a label is incorporated in the target nucleic acid or probe by incorporation labeling, e.g., using a hapten of a modified nucleotide that is recognized by an antibody.

The term “CYP2D6 target sequence” refers to a sequence in a nucleic acid molecule corresponding to CYP2D6 that it is desired to detect. The sequence may be in any nucleic acid sequence corresponding to a CYP2D6 gene, e.g., in a coding sequence, an intron, a 3′ untranslated region, or a 5′ untranslated region. Such 5′ and 3′ sequences can include, for example, promoter and enhancer sequences. Generally, target sequences for this invention are sequences including or near (preferably within 10, 40, 60, or 100 nucleotides of) an identified polymorphic site. Typically such a target sequence is a complementary sequence for a probe or amplification oligonucleotide, such as a PCR primer. Preferably a target sequence includes a polymorphic site. Thus, a probe or a primer can be used to distinguish from a target sequence and a non-target sequence that differ at the polymorphic sites using differential hybridization or differences in extension or amplification efficiency. Similarly, “target sequence” can be used to refer to nucleic acid sequences corresponding to other genes, preferably a gene listed herein.

A nucleic acid sequence or molecule is “corresponding” to a particular gene, e.g., CYP2D6 gene, if it is part of or is derived from that gene, a complementary sequence, or an RNA equivalent of such a sequence. Thus, for example, an mRNA or portion thereof, a genomic sequence or portion thereof, a cDNA sequence or portion thereof, and sequences complementary to such sequences all correspond to a particular gene. In the case of a portion, the portion is of sufficient length to distinguish the portion from other nucleic acid sequences that may be present.

The term “polymorphic site” refers to a location in a nucleic acid sequence that is known to differ in sequence between individuals. As understood by those skilled in the art, a number of different polymorphisms may be involved, including single nucleotide substitutions, deletions of one or more nucleotides (which may result in a frame-shift), and insertions of one or more nucleotides (which also may result in a frame-shift). Among the most common polymorphisms are single nucleotide polymorphisms (SNPs). Thus, for example, for use of an allele-specific probe, a SNP will generate a single base mismatch as compared to wild type, while a deletion can generate a mismatch or even the absence of a binding sequence for a particular probe. Likewise, insertions can generate mismatches or insertion or relocation of target sequences.

In the context of nucleic acid hybridizations for this invention, the term “stringent conditions” refers to conditions that are sufficiently restrictive as to provide distinguishably different levels or stabilities of hybridization for a particular nucleic acid sequence of interest as compared to other nucleic acid sequences that may be present in a sample is sufficient numbers to potentially provide hybridization that would interfere with determination of specific hybridization.

In another aspect, the invention provides an amplification oligonucleotide primer, e.g., a PCR primer or other amplification oligonucleotide, adapted for amplifying a portion of a gene, e.g., CYP2D6 gene, including a sequence polymorphism. In particular embodiments, the oligonucleotide binds to an intron of the gene, e.g., CYP2D6 gene. Also in particular embodiments, the primer is a gene-specific primer and/or an allele-specific primer, which may bind to an intron or to an exon. Preferably the oligonucleotide is a PCR primer. Similarly included are extension primers.

The primer or other amplification oligonucleotide preferably hybridizes under stringent hybridization conditions to a sequence corresponding to a gene, e.g., target site, CYP2D6 so that the primer contains at least one nucleotide at the 3′ end that base pairs with a complementary nucleotide in a target sequence in at least one allele and does not base pair with a complementary nucleotide in a target sequence in at least one different allele of the gene. Thus, amplification will preferentially occur in the presence of a particular sequence at a polymorphic site as compared to a different sequence at that site.

The present invention includes the identification of particular useful PCR primers. Those primers have sequences as provided in the Examples.

Preferably the primers preferentially extend and/or amplify nucleic acid corresponding to the gene of interest in preference to a pseudo-gene or expressed non-functional gene with related sequence, e.g., extend and/or amplify CYP2D6 in preference to CYP2D7 and/or CYP2D8.

In connection with primers, the term “gene-specific” indicates that the primer preferentially binds to, and/or extends or amplifies a sequence corresponding to a particular gene in preference to sequences corresponding to other genes that may be present in a sample to a sufficiently greater extent as to allow distinguishing the amplified or extended products corresponding to the particular gene. Preferably no appreciable amplification will occur for other genes present.

Similarly, in connection with primers, the term “allele-specific” means that the primer preferentially binds to, and/or extends or amplifies a sequence corresponding to a particular form (or subset of forms) of a polymorphism in a gene, in preference to sequences corresponding to other forms of the polymorphism in that gene to a sufficiently greater extent as to allow distinguishing the amplified or extended products corresponding to the particular form. Preferably no appreciable amplification will occur for other forms of the polymorphism present.

In connection with binding and/or amplification, the term “preferentially” indicates that the process occurs to a greater extent with a particular substrate that with some other substrate(s). Thus, for example, a gene-specific probe binds to the appropriate target to a greater extent and/or with greater stability than to other nucleotide sequences that may be present in the sample. The greater extent of binding (or greater stability) and/or amplification is sufficiently greater to allow discrimination of the dominant process from the less frequently occurring or less stable process. For example, with gene-specific amplification, amplification of non-target sequences may be undetectable in an assay.

In connection with amplification oligonucleotides, the phrase “adapted for amplifying” indicates that the sequence is selected to provide appropriate binding stability, location, and other characteristics suitable to provide useful amplification of a particular substrate sequence under amplification conditions.

As the presence of particular sequence variants can also be identified using allele specific probes, in another aspect, the invention provides at least one allele specific probe, preferably a designed set of nucleic acid probes complementary to a target nucleic acid sequence or sequences, e.g., a CYP2D6 target nucleic acid sequence or sequences. Such a probe is or includes a molecule that preferentially binds to a target nucleic acid sequence, e.g., a CYP2D6 target nucleic acid sequence, at least partially including a sequence polymorphism in a particular gene. A scattered light detectable particle 1-500 nm in size, preferably a gold, silver, or mixed gold and silver particle, is bound with the target. The particle may be directly or indirectly bound. Thus, the particle may be attached to the target using a link between the particle or a coating on the particle. Alternatively, the particle may be attached to the target using a separate binding pair, such as nucleic acid hybridization, biotin/avidin or streptavidin, antigen/antibody (e.g., biotinlanti-biotin), or other binding pair interaction. Thus, in certain embodiments, the particle may be bound to the target at the time the target binds a probe sequence, or may be attached to the target after the target is bound to the probe sequence.

In certain embodiments, the nucleic acid probe has a polyA tail, preferably 5-50 nucleotides in length. Alternatively, the tail may be 10-50, 10-40, 10-30, 20-50, 20-40, or 20-30 nucleotides in length.

The invention also provides one or more isolated nucleic acid sequences corresponding to a gene, e.g., CYP2D6. Each such sequence is bound with a probe, preferably an allele-specific probe, and a scattered light detectable particle. As indicated above, the particle may be bound directly or indirectly to the target. Thus, the scattered light detectable particle can be bound to a first member of a binding pair, where the first member of a binding pair is bound with the second member of the binding pair; and the second member of the binding pair is bound with the probe.

Preferably, there are a plurality of different sequences corresponding to a particular gene, e.g., CYP2D6. In preferred embodiments, there are a plurality of distinguishably different particles, bound respectively with different nucleic acid sequences.

The invention also provides a method for determining the presence of an allele in a particular gene, e.g., a CYP2D6 allele, in a nucleic acid sample that may contain nucleic acid corresponding to the gene, by contacting the nucleic acid sample with at least one allele-specific probe under conditions wherein the probe or probes specifically bind to any nucleic acid target corresponding to the gene in the sample that includes a specific sequence polymorphism, and not to (or to a detectably lesser extent and/or stability) nucleic acid corresponding to the gene that does not include the specific sequence polymorphism. The target or a sequence-specific probe is bound with at least one scattered-light detectable particle of a size between 1 and 500 nm inclusive. The method also involves illuminating any such particles bound with probe bound and/or with nucleic acid corresponding to a particular gene, such as CYP2D6, with light under conditions which produce scattered light from the particles and in which light scattered from one or more particles can be detected; and detecting light scattered by any such particles under those conditions as a measure of the presence of the nucleic acid corresponding to the gene including said specific sequence polymorphism.

Preferably the methods of this invention use illumination with non-evanescent wave light, and the scattered light can be detected by a human eye with less than 500 times magnification and without electronic amplification. However, other detection methods may be used as known to those skilled in the art.

Preferably the probe includes a nucleic acid sequence that hybridizes with the nucleic acid corresponding to CYP2D6.

As in the method described above, target nucleic acid or probe may be labeled. The labeling can be inserted by incorporation labeling, e.g., as a hapten or a modified nucleotide that is recognized by an antibody.

In another aspect, the invention provides a method for detecting the presence or absence of specific polymorphisms or alleles of a gene, preferably CYP2D6, by amplifying a portion or portions of the gene using one or more of the specific primers described above, and detecting the presence or absence of amplified nucleic acid sequence or of a target sequence within amplified nucleic acid sequence as an indication of the presence or absence of the specific polymorphism(s).

In preferred embodiments, the detection is carried out as described for other aspects herein, using scattered light detectable particles as detectable labels.

In another aspect, the invention provides a kit adapted for determination of the presence of at least one sequence polymorphism in target nucleic acid corresponding to a gene, preferably CYP2D6. The kit includes at least one array chip, where the array chip is adapted to bind target nucleic acid at a plurality of spots under binding conditions suitable for discriminating binding to target sequences from non-target sequences. For example, binding can be discriminated between a mutant sequence and a wild type sequence. The kit includes at least one allele specific probe that specifically binds to a target sequence preferably CYP2D6, and at least one scattered light detectable particle 1 to 500 nm in size that binds to the nucleic acid. The allele specific probe can be capture probe or detection probe.

In preferred embodiments the array chip is adapted to bind a plurality of different target nucleic acid molecules, e.g., CYP2D6 at different spots. Such a plurality of spots can be a number as described above.

Also in preferred embodiments, the at least one allele-specific probe comprises a plurality of different allele-specific probes. The plurality of different probes may be, for example, 2, 3, 4, 5, 6, 7, 8, 9, 10 or more.

Similarly, in another aspect, the invention provides a kit adapted for determination of the presence of at least one sequence polymorphism in target nucleic acid preferably corresponding to CYP2D6, which includes at least one allele-specific probe that specifically binds to a target sequence, and at least one scattered light detectable particle adapted to bind with the allele-specific probe or target sequence. Preferably the kit also includes at least one array chip containing nucleic acid molecules corresponding to a particular gene, e.g., CYP2D6, e.g., capture probes for target CYP2D6 nucleic acid.

In preferred embodiments, the at least one allele-specific probe includes a plurality of different allele-specific probes.

In preferred embodiments, the at least one scattered light detectable particle is bound to the at least one allele-specific probe. Typically a single particle is attached to a single probe molecule, though it is possible to attach multiple particles, e.g., 2, 3, or more. The at least one particle can be a plurality of different particles, where the different particles have distinguishable light scattering particles. An example of such different light scattering particles is different colors of scattered light on illumination with polychromatic light, such as white light.

The kit can also include at least one, and preferably a plurality of, amplification oligonucleotide adapted to bind to or extend through a polymorphic site, e.g., a CYP2D6 polymorphic site. For example, the oligonucleotide(s) may be PCR primers, oligonucleotides for non-PCR amplification, or primers for non-amplification extension reactions.

The kits of this invention may also advantageously include other components, such as one or more of suitable buffers for hybridization, buffers for DNA synthesis, wash solutions, nucleoside triphosphates, and light scattering particle suspensions.

Also in preferred embodiments, the kit is packaged in a single container, with particular components held separately therein. Preferably the kit also includes a set of instructions for use, describing how to perform the test and/or how to interpret results.

In connection with kits, the term “adapted for” indicates that the kit components are designed and selected to provide the specified function at a useful level. This is distinguished, for example, from a collection of items that could merely be utilized to provide some minimal level of function, or from which a subset of items could be selected that could provide a function specified for the present invention, but that also contains a variety of other components unrelated to the present invention. For example, a set of random oligonucleotides, in which one oligonucleotide, by chance, had a sequence that would allow the oligo to function as a probe, would not constitute a kit.

If desired, in embodiments of this invention, gene or gene fragment sequence determinations or target sequence determinations can be performed on any nucleic acid sequence corresponding to a gene of interest, e.g., CYP2D6 gene, including genomic DNA, cDNA, mRNA, or other RNA, and nucleic acid sequences complementary thereto. Such sequencing can be performed, for example, by any of a variety of sequencing methods known in the art.

In the context of a connection between a probe and a light scattering particle, the term “associated with” refers to a direct or indirect binding interaction. For example, the light scattering particle may be physically attached to the probe, to the probe-target complex or a complex that includes probe and target, or to another component that binds with the probe or probe-target complex or complex that includes the probe and target. In certain embodiments, the light scattering particle is directly or indirectly bound either before a sample is contacted with the probe; in other embodiments the light scattering particle is bound to the probe or complex subsequent to such contact.

Preferably, genes are selected for use in this invention are pharmacogenetically relevant genes, i.e., clinically relevant genes with sequence polymorphisms that affect the treatment, course, development, or serenity of a disease or condition.

In addition to CYP2D6, additional specific genes of particular interest for use in the present invention include the human genes CYP2C19, CYP2C9, NAT-2, IRF-1, RANTES, and VEGF.

In particular embodiments of the aspects of the present invention, methods and/or particles and/or apparatus as described in any of Yguerabide et al. PCT/US97/06584, Yguerabide et al., PCT/US98/23160, Yguerabide et al, U.S. Pat. No. 6,214,560, and Yguerabide et al., U.S. application Ser. No. 08/953,713 are utilized.

Also, in particular embodiments of the aspects of the present invention, target nucleic acid is prepared using enzymatic incorporation labeling. Such labeling can be accomplished in various ways, all within the present invention. The incorporation labeling can be carried out with exponential target amplification, including PCR methods and non-PCR methods, e.g., ligase chain reaction. The incorporation labeling can also be carried out with non-exponential, low level amplification. For example, in particular embodiments, the amplification is no more than 50-fold, 20-fold, 10-fold, 5-fold, or 2-fold. Such low level amplification can be accomplished, for example, using primer extension reactions, with multiple rounds of binding and extension. Incorporation can also be carried out without amplification, for example, with simple extension reactions without cycling or multiple rounds of binding and extension. A number of different incorporation labeling techniques can be utilized, e.g., techniques pointed out herein.

Also, as indicated, the label incorporated can be of different types, for example, incorporation of a hapten, allowing binding with a binding molecule, e.g., incorporation of biotin, allowing binding of avidin or streptavidin. Another example is incorporation of a modified nucleotide that provides antibody binding. Alternatively, the modified nucleotide can provide a location for chemical or physical cleavage or a site for chemical modification, e.g., a particular reactive moiety that allows modification that particular site in preference to other sites in the molecule.

Targets can also be prepared by chemical labeling.

By “incorporation labeling” is meant that a moiety is included in a nucleic acid molecule during synthesis that provides for direct or indirect binding of a detectable label not requiring nucleic acid hybridization for binding the detectable label. For example, the incorporated label may itself be a detectable label, or may provide a site for binding of another molecule, e.g., hapten or antibody binding., or may provide a site for chemical modification.

Thus, the present invention includes a target molecule(s) corresponding to a portion of a gene, where that portion includes a polymorphic site (or a portion thereof in cases where the polymorphism involves an extended insertion or deletion), e.g., a SNP site. The target molecule is prepared using incorporation labeling, e.g., in a manner as described above, and thus included such an incorporated label, or is labeled using chemical labeling, and thus includes such a chemically introduced label. An example of such a target is a sequence corresponding to CYP2D6 or other target genes indicated herein.

The invention also includes probes, primers (and other amplification oligonucleotides), and/or target molecules that are directly or indirectly bound to RLS particles, preferably as described in any of Yguerabide et al. PCT/US97/06584, Yguerabide et al., PCT/US98/23160, Yguerabide et al, U.S. Pat. No. 6,214,560, and Yguerabide et al., U.S. application Ser. No. 08/953,713. Examples include primers, probes, and target molecules corresponding to CYP2D6 or other target gene indicated herein, preferably corresponding to a polymorphic site in such gene.

The invention further provides methods for preparing labeled targets using incorporation labeling, e.g. as described herein. The methods can also include directly or indirectly binding the targets with RLS particles.

The present invention can be applied to nucleic acid molecules corresponding to any genomic DNA, but preferably corresponding to gene sequences and/or mammalian DNA. More preferably the invention is applied to human genes, most preferably to pharmacogenetically relevant genes, such as those involved in drug metabolism, modification, and/or excretion. CYP2D6 (human) is an example of such a gene.

In addition, preferably a polymorphism to be detected has been demonstrated to be pharmacogenetically relevant.

In connection with genes and polymorphisms, the term “pharmacogenetically relevant” means that the gene or polymorphism has been demonstrated to affect the risk of acquiring or developing a disease or pathological condition, the course or severity, or the probability of a course or severity of a disease or other pathological condition, a response or probability of a response of a disease or other pathological condition to a treatment, or the ability or probability of the ability of a mammal, e.g., a human, to tolerate a treatment. Examples of treatment include administration of a drug, administration of radiation, and medically-based modification of lifestyle, such as dietary modification. Thus, such genes are particularly relevant to pathological conditions, and are distinguished from genes that affect the structure and function of a mammal during normal condition but do not have an added particular significance in development and/or treatment of a pathological condition. Similarly, some polymorphisms, even in pharmacogenetically relevant genes are not pharmacologically relevant. Examples include genes that are pharmacologically relevant because the encoded protein is pharmacogenetically relevant, but the polymorphism at the nucleic acid level does not result in a change in amino acid sequence, or the polymorphism results in an amino acid change, but that change does not correlate with any of the indicators of pharmacogenetic relevance. Preferably a polymorphism or a set of polymorphisms, e.g., 2, 3, 4, 5, 6, 8, 10, or even more polymorphisms in a gene account for at least 10% of the variation in treatment response or other pharmacogenetic indicator, more preferably at least 20%, 30%, 40%, 50%, 60%, 70%, 80%, or 90% of that variation.

Additional embodiments will be apparent from the Detailed Description and from the claims.

### BRIEF DESCRIPTION OF THE DRAWINGS

FIG. 1 is a schematic description of a random primer incorporation for incorporation labeling.

FIG. 2 is a schematic diagram of a nick translation method for incorporation labeling.

FIG. 3 is a schematic diagram of a biased primer extension method for incorporation labeling.

FIG. 4 is a schematic diagram of a gene-specific primer extension method for incorporation labeling.

FIG. 5 is an extension displacement transcription incorporation method for incorporation labeling.

FIG. 6 lists exemplary probes for CYP2D6 allele detection.

FIG. 7 shows relative positions of CYP2D6 probes and primers useful for allele determinations.

### DETAILED DESCRIPTION OF THE PREFERRED EMBODIMENTS

A. Introduction

The present invention is directed to determination of the presence of particular sequence variances in a gene. The invention is described herein principally with respect to the preferred exemplary gene, CYP2D6. However, one of ordinary skill in the art will recognize that the aspects of the present invention can be generally applied to a multitude of other genetic polymorphic systems to develop simple and sensitive assays for SNP detection. For example, the invention can be applied to a gene involved in drug metabolism and other detoxification processes, or other gene identified herein. In connection with genes involved in drug metabolism, one pharmacokinetic consequence of drug metabolism within the human body is to make drug entities more water soluble, thereby facilitating excretion via urine or bile. The cytochrome P450 enzymes (CYP) are a select family of enzymes found mainly in the liver and provide one method of metabolizing drugs by altering the functional groups on the parent molecule. Each enzyme is derived from a different gene and thus is termed an isoform.

Cytochrome P450 (CYP450) is comprised of a large family of proteins that are of central importance to the detoxification or activation of numerous foreign hydrophobic entities, including many therapeutic drugs. The CYP2D subfamily, and in particular the CYP2D6 isoenzyme is a monoxygenase responsible for the primary metabolism of debrisoquine and dextramethorphan, as well as a number of beta-blockers, anti-psychotics and anti-depressants. Current data shows that 7% to 10% of the Caucasian population have specific mutations (or polymorphisms) within the CYP2D6 gene that result in a reduced activity of the enzyme. These poor metabolizers (PM) are at a higher risk for drug accumulation and toxicity as well as a reduction in efficacy if the active moiety of a compound is the metabolite of a CYP2D6 substrate (i.e. the breakdown of codeine to morphine). Current methods for the detection of specific CYP2D6 mutations utilize PCR-coupled fluorescence or gel electrophoresis.

An exemplary assay of the present invention utilizes PCR, resonance light scattering (RLS) particles and nucleic acid hybridization technology to analyze the genotype of certain CYP2D6 mutations on a single slide.

B. CYP2D6 Polymorphism Identification and Allele Characterization

As indicated above, polymorphisms or mutations in the CYP2D6 gene significantly affect the function of the gene product, and therefore affect the metabolism of molecules, such as a number of different therapeutic drugs, that are normally modified, or metabolized, by CYP2D6. The level of function of various alleles results in phenotypic classifications of individuals based on the metabolizing activity of the gene product.

The CYP2D6 isoform exhibits a large number of genetic polymorphisms. There are currently 38 known alleles of the CYP2D6 gene as shown in a table below. These alleles encode for varying functionality of the CYP2D6 protein. The resulting phenotypic expression can be classified into one of four groups; (1) Ultrarapid Metabolizer (UM), (2) Extensive Metabolizer (EM), (3) Intermediate Metabolizer (IM) and (4) Poor Metabolizer (PM) (Table 1). These varying enzymatic functions and subsequent phenotypic classifications can be confirmed utilizing a number of well-characterized systems based on the metabolic ratio (MR) of CYP2D6 regulated compounds (Table 2). The classifications are based on the following criteria. (Unless otherwise indicated, phenotypic classification herein are based on metabolism of debrisoquine to 4-hydroxydebrisoquine)

Ultrarapid Metabolizer—Metabolic Ratio<0.1-0.2

Extensive Metabolizer—Normal Metabolic Ratio

Intermediate Metabolizer—Metabolic Ratio>5.4

Poor Metabolizer—Metabolic Ratio>12.6

The classifications are also shown in the table below.

TABLE 1
CYP2D6 Phenotypic Classification Definitions
Ultra-rapid Metabolizer (UM):
An individual that carries at least 3 functional copies of the CYP2D6
gene (CYP2D6*2XN/*1)
Extensive Metabolizer (EM):
An individual that carries 1 or 2 functional copies of the CYP2D6 gene
(CYP2D6*1/*1)
Intermediate Metabolizer (IM):
An individual that carries 1 allele associated with diminished CYP2D6
activity (CYP2D6*2, *9, *10, 17) and 1 non-functional CYP2D6 allele
(CYP2D63, *4, *5, *6, *7, *8, *11)
Poor Metabolizer (PM):
An individual that carries any 2 non-functional CYP2D6 alleles
(CYP2D6*3, *4, *5, *6, *7, *8, *11)

TABLE 2

| | |
|---|---|
| Enzyme Activity/Phenotype | Metabolic Ratio (MR) |

Metabolic Ratio of debrisoquine to 4-hydroxydebrisoquine

| | |
|---|---|
| Ultrarapid Metabolizer | MR ≦ 0.2 |
| Extensive Metabolizer | 1.0 to 4.0 |
| Intermediate Metabolizer | 5.4 to 10.0 |
| Poor Metabolizer | MR ≧ 12.6 |

Metabolic Ratio of dextromethorphan to dextrophan

| | |
|---|---|
| Ultrarapid Metabolizer | MR ≦ 0.003 |
| Extensive Metabolizer | MR ≈ 0.005 |
| Intermediate Metabolizer | MR ≈ 0.018 |
| Poor Metabolizer | MR ≦ 3.00 |

The incidence of poor metabolizers varies among different populations. The prevalence of the PM phenotype has been shown to range between 19% for certain black populations to 1% for some Oriental populations. Interracial differences are attributed to an unequal distribution of the CYP2D6 alleles among different populations. For Caucasians, it has been shown that 10% of the population can be classified, with regards to the CYP2D6 gene, as PM. This poor metabolizer phenotype can result in a higher risk for drug accumulation and toxicity as well as a reduction in efficacy if the active moiety is a metabolite of a CYP2D6 regulated compound. A exemplary list of clinically available compounds that are substrates of the CYP2D6 pathway and could pose a direct risk to poor metabolizers are provided in Table 3.

TABLE 3
Clinically Available Drugs That Are Substrates Of CYP2D6

| Beta Blockers | Antidepressants | Antipsychotics | Others |
|---|---|---|---|
| S-metoprolol | Amitriptylline | haloperidol | Codeine |
| propafenone | Clomipramine | risperidone | Dextromethorphan |
| timolol | Desipramine | thioridazine | Flecainide |
| | Imipramine | | Ondansetron |
| | | | Tramadol |
| | | | Venlafaxine |

For those Caucasians classified as poor metabolizers, nearly 95% are accounted for by 4 specific alleles. These alleles are CYP2D6*3, CYP2D6*4, CYP2D6*6 and CYP2D6*7. In Chinese populations, though, the CYP2D6*3 and *4 alleles are rarely found but the prevalence of the CYP2D6*10a or *10b allele is nearly 50%. Although the CYP2D6*10 allele encodes for the Intermediate Metabolizer phenotype, it emphasizes the genetic variation inherent in different populations.

The locations of mutations for alleles utilized in an exemplary assay are shown in the table below, along with a number of additional mutations. Such additional mutations can be utilized in other tests.

CYP2D6 allele nomenclature

| | | Nucleotide | XbaI haplo- | Trivial | | Enzyme activity | | |
|---|---|---|---|---|---|---|---|---|
| Allele | Protein | changes | type (kb) | name | Effect | In vivo | In vitro | References |
| CYP2D6*1A | CYP2D6.1 | None | 29 | Wild- | | Normal | Normal | Kimura et |
| | | | | type | | | | al, 1989 |
| CYP2D6*1B | CYP2D6.1 | 3828G > A | 29 | | | Normal | | Marez et |
| | | | | | | (d, s) | | al, 1997 |
| CYP2D6*1C | CYP2D6.1 | 1978C > T | | M4 | | Normal | | Marez et |
| | | | | | | (s) | | al, 1997 |
| CYP2D6*1D | CYP2D6.1 | 2575C > A | | M5 | | | | Marez et |
| | | | | | | | | al, 1997 |
| CYP2D6*1E | CYP2D6.1 | 1869T > C | | | | | | Sachse et |
| | | | | | | | | al, 1997 |
| CYP2D6*1XN | CYP2D6.1 | | 42 | | N active | Incr | | Dahl et al, |
| | | | | | genes | | | 1995 |
| | | | | | | | | Sachse et |
| | | | | | | | | al, 1997 |
| CYP2D6*2A | CYP2D6.2 | 1661G > C; | 29 | CYP2 | R296C; | (Decr) | (Decr) | Johansson |
| | | 2850C > T; | | D6L | S486T | (dx,d) | | et al, 1993 |
| | | 4180G > C | | | | | | Panserat et |
| | | | | | | | | al, 1994 |
| CYP2D6*2B | CYP2D6.2 | 1039C > T; | | | R296C; | | | Marez et |
| | | 1661G > C; | | | S486T | | | al, 1997 |
| | | 2850C > T; | | | | | | |
| | | 4180G > C | | | | | | |
| CYP2D6*2C | CYP2D6.2 | 1661G > C; | | | R296C; | | | Marez et |
| | | 2470T > C; | | | S486T | | | al, 1997 |
| | | 2850C > T; | | | | | | Sachse et |
| | | 4180G > C | | | | | | al, 1997 |
| CYP2D6*2D | CYP2D6.2 | 2850C > T; | | M10 | R296C, | | | Marez at |
| | | 4180G > C | | | S486T | | | al, 1997 |
| CYP2D6*2E | CYP2D6.2 | 997C > G; | | M12 | R296C, | | | Marez et |
| | | 1661G > C; | | | S486T | | | al, 1997 |
| | | 2850C > T; | | | | | | |
| | | 4180G > C | | | | | | |
| CYP2D6*2F | CYP2D6.2 | 1661G > C; | | M14 | R296C; | | | Marez et |
| | | 1724C > T; | | | S486T | | | |
| | | 2850C > T; | | | | | | |
| | | 4180G > C | | | | | | |
| CYP2D6*2G | CYP2D6.2 | 1661G > C; | | M16 | R296C; | | | Marez et |
| | | 2470T > C; | | | S486T | | | al, 1997 |
| | | 2575C > A; | | | | | | |
| | | 2850C > T; | | | | | | |
| | | 4180G > C | | | | | | |
| CYP2D6*2H | CYP2D6.2 | 1661G > C; | | M17 | R296C; | | | Marez at |
| | | 2480C > T; | | | S486T | | | al, 1997 |
| | | 2850C > T; | | | | | | |
| | | 4180G > C | | | | | | |
| CYP2D6*2J | CYP2D6.2 | 1661G > C; | | M18 | R296C; | | | Marez et |
| | | 2850C > T; | | | S486T | | | al, 1997 |
| | | 2939G > A; | | | | | | |
| | | 4180G > C | | | | | | |
| CYP2D6*2K | CYP2D6.2 | 1661G > C; | | M21 | R296C; | | | Marez |
| | | 2850C > T; | | | S486T | | | al, 1997 |
| | | 4115C > T; | | | | | | |
| | | 4180G > C | | | | | | |
| CYP2D6*2XN | CYP2D6.2 | 1661G > C; | 42-175 | | R296C; | Incr | | Johansson |
| (N = 2, 3, 4, 5 | | 2850C > T; | | | S486T | (d) | | et al, 1993. |
| or 13) | | 4180G > C | | | N active | | | Dahl et al, |
| | | | | | genes | | | 1995 |
| | | | | | | | | Aklillu at |
| | | | | | | | | al, 1996 |
| CYP2D6*3A | | 2549A > del | 29 | CYP2 | Frame- | None | None | Kagimoto |
| | | | | D6A | shift | (d, s) | (b) | et al, 1990 |
| CYP2D6*3B | | 1749A > G; | | | N166D; | | | Marez et |
| | | 2549A > del | | | frame- | | | al, 1997 |
| | | | | | shift | | | |
| CYP2D6*4A | | 100C > T; | 44, 29, 16 + 9 | CYP2 | P34S; | None | None | Kagimoto |
| | | 974C > A; | | D6B | L91M; | (d, s) | (b) | et al, 1990 |
| | | 984A > G; 99 | | | H94R; | | | Gough et |
| | | 7C > G; | | | Splicing | | | al, 1990 |
| | | 1661G > C; | | | defect; | | | Hanioka et |
| | | 1846G > A; | | | S486T | | | al, 1990 |
| | | 4180G > C | | | | | | |
| CYP2D6*4B | | 100C > T; | 29 | CYP2 | P34S; | None | None | Kagimoto |
| | | 974C > A; | | D6B | L91M; | (d, s) | (b) | et al, 1990 |
| | | 984A > G; | | | H94R; | | | |
| | | 997C > G; | | | Splicing | | | |
| | | 1846G > A; | | | defect, | | | |
| | | 4180G > C | | | S486T | | | |
| CYP2D6*4C | | 100C > T; | 44/29 | K29-1 | P345; | None | | Yokota et |
| | | 1661G > C; | | | Splicing | | | al, 1993 |
| | | 1846G > A; | | | defect; | | | |
| | | 3887T > C; | | | L421P; | | | |
| | | 4180G > C | | | S486T | | | |
| CYP2D6*4D | | 100C > T; | | | P34S; | None | | Marez et |
| | | 1039C > T; | | | Splicing | (dx) | | al, 1997 |
| | | 1661G > C; | | | defect; | | | |
| | | 1846G > A; | | | S486T | | | |
| | | 4180G > C | | | | | | |
| CYP2D6*4E | | 100C > T; | | | P34S; | | | Marez et |
| | | 1661G > C; | | | Splicing | | | al, 1997 |
| | | 1846G > A; | | | defect; | | | |
| | | 4180G > C | | | S486T | | | |
| CYP2D6*4F | | 100C > T; | | | P34S; | | | Marez et |
| | | 974C > A; | | | L91M; | | | al, 1997 |
| | | 984A > G; | | | H94R; | | | |
| | | 997C > G; | | | Splicing | | | |
| | | 1661G > C; | | | defect; | | | |
| | | 1846G > A; | | | R173C; | | | |
| | | 1858C > T; | | | S486T | | | |
| | | 4180G > C | | | | | | |
| CYP2D6*4G | | 100C > T; | | | P34S; | | | Marez et |
| | | 974C > A; | | | L91M; | | | al, 1997 |
| | | 984A > G; | | | H94R; | | | |
| | | 997C > G; | | | Splicing | | | |
| | | 1661G > C; | | | defect; | | | |
| | | 1846G > A; | | | P325L; | | | |
| | | 2938C > T; | | | S486T | | | |
| | | 4180G > C | | | | | | |
| CYP2D6*4H | | 100C > T; | | | P34S; | | | Marez et |
| | | 974G > A; | | | L9LM; | | | al, 1997 |
| | | 984A > G; | | | H94R; | | | |
| | | 997C > G; | | | Splicing | | | |
| | | 1661G > C; | | | defect; | | | |
| | | 1846G > A; | | | E418Q; | | | |
| | | 3877G > C; | | | S486T | | | |
| | | 4180G > C | | | | | | |
| CYP2D6*4J | | 100C > T; | | | P34S; | | | Marez et |
| | | 974C > A; | | | L91M; | | | al, 1997 |
| | | 984A > G; | | | H94R; | | | |
| | | 997C > G; | | | Splicing | | | |
| | | 1661G > C; | | | defect | | | |
| | | 1846G > A | | | | | | |
| CYP2D6*4K | | 100C > T; | | | P34S; | None | | Sachse et |
| | | 166 1G > C; | | | Splicing | | | al, 1997 |
| | | 1846G > A; | | | defect; | | | |
| | | 2850C > T; | | | R296C; | | | |
| | | 4180G > C | | | S486T | | | |
| CYP2D6*4X2 | | | 32 + 9 | | | None | | Løvlie et |
| | | | | | | | | al, 1997 |
| | | | | | | | | Sachse et |
| | | | | | | | | al, 1998 |
| CYP2D6*5 | | CYP2D6 | 11.5 or 13 | CYP2 | CYP2D6 | None | | Gaedigk et |
| | | deleted | | D6D | deleted | (d, s) | | al, 1991 |
| | | | | | | | | Steen et al, |
| | | | | | | | | 1995 |
| CYP2D6*6A | | 1707T > del | 29 | CYP2 | Frame- | None | | Saxena et |
| | | | | D6T | shift | (d, dx) | | al, 1994 |
| CYP2D6*6B | | 1707T > del; | 29 | | Frame- | None | | Even et al, |
| | | 19760 > A | | shift; | (s, d) | | 1994 | |
| | | | | | G212E | | | Daly et al, |
| | | | | | | | | 1995 |
| CYP2D6*6C | | 1707T > del; | | | Frame- | None (s) | | Marez et |
| | | 1976G > A; | | | shift; | | | al, 1997 |
| | | 4180G > C | | | G212E; | | | |
| | | | | | S486T | | | |
| CYP2D6*6D | | 1707T > del; | | | Frame- | | | Marez et |
| | | 32880 > A | | | shift; | | | al, 1997 |
| | | | | | G373S | | | |
| CYP2D6*7 | CYP2D6.7 | 2935A > C | 29 | CYP2 | H324P | None | | Even et al, |
| | | | | D6E | | (s) | | 1994 |
| CYP2D6*8 | | 1661G > C; | | CYP2 | Stop | None | | Broly at al, |
| | | 1758G > T; | | D6G | codon; | (d, s) | | 1995 |
| | | 2850C > T; | | | R296C; | | | |
| | | 41800 > C | | | S486T | | | |
| CYP2D6*9 | CYP2D6.9 | 2613- | 29 | CYP2 | K281del | Decr | Decr | Tyndale et |
| | | 261SdelAGA | | D6C | | (b,s,d) | (b,s,d) | al, 1991 |
| | | | | | | | | Broly & |
| | | | | | | | | Meyer, |
| | | | | | | | | 1993 |
| CYP2D6*10A | CYP2D6.10 | 100C > T; | 44, 29 | CYP2 | P34S; | Decr | | Yokota et |
| | | 1661G > C; | | D6J | S486T | (s) | | al, 1993 |
| | | 4180G > C | | | | | | |
| CYP2D6*10B | CYP2D6.10 | 100C > T; | 44, 29 | CYP2 | P34S; | Decr | Decr | Johansson |
| | | 1039C > T; | | D6Chl | S486T | (d) | (b) | et al, 1994 |
| | | 1661G > C; | | | | | | |
| | | 4180G > C | | | | | | |
| CYP2D6*10C | see CYP2D6*36 | | | | | | | |
| CYP2D6*11 | | 883G > C; | 29 | CYP2 | Splicing | None | | Marez et |
| | | 1661G > C; | | D6F | defect; | (s) | | al, 1995 |
| | 28500 > T; | | | R296C; | | | | |
| | 4180G > C | | | S486T | | | | |
| CYP2D6*12 | CYP2D6.12 | 124G > A; | 29 | | G42R;; | None | | Marez et |
| | | 16610 > C; | | | R296C; | (s) | | al, 1996 |
| | | 2850C > T; | | | S486T | | | |
| | | 41800 > 0 | | | | | | |
| CYP2D6*13 | | CYP2D7P/CY | 29 | | Frame- | None | | Panserat et |
| | | P2D6 hybrid. | | | shift | (dx) | | al, 1995 |
| | | Exon 1 | | | | | | |
| | | CYP2D7, | | | | | | |
| | | exons 2-9 | | | | | | |
| | | CYP2D6. | | | | | | |
| CYP2D6*14 | CYP2D6.14 | 100C > T; | 29 | | P34S; | None | | Wang. |
| | | 1758G > A; | | | G169R; | (d) | | 1992 |
| | | 2850C > T; | | | R296C; | | | Wang et al, |
| | | 41800 > C | | | S486T | | | 1999 |
| CYP2D6*15 | | 138insT | 29 | | Frame- | None | | Sachse et |
| | | | | | shift | (d, dx) | | al, 1996 |
| CYP2D6*16 | | CYP2D7P/CY | 11 | CYP2 | Frame- | None | | Daly et al, |
| | | P2D6 hybrid. | | D6D2 | shift | (d) | | 1996 |
| | | Exons 1-7 | | | | | | |
| | | CYP2D7P- | | | | | | |
| | | related, exons | | | | | | |
| | | 8-9 CYP2D6. | | | | | | |
| CYP2D6*17 | CYP2D6.17 | 1023 C > T; | 29 | CYP2 | T107I; | Decr | Decr | Masimirembwa |
| | | 1638G > C: | | D6Z | R296C; | (d) | (b) | et al, |
| | | 2850C > T; | | | S486T | | | 1996 |
| | | 4180G > C | | | | | | Oscarson |
| | | | | | | | | et al, 1997 |
| CYP2D6*18 | CYP2D6.18 | 9 bp insertion | 29 | CYP2 | | Decr (s) | Decr (b) | Yokoi et |
| | | in exon 9 | | D6(J9) | | | | al, 1996 |
| CYP2D6*19 | | 1661G > C; | | | Frame- | None | | Marez et |
| | | 2539- | | | shift; | | | al, 1997 |
| | | 2542delAAC | | | R296C; | | | |
| | | T; 2850C > T; | | | S486T | | | |
| | | 4180G > C | | | | | | |
| CYP2D6*20 | | 1661G > C; | | | Frame- | None | | Marez |
| | | 1973insG;19 | | | shift; | (m) | | Allorge et |
| | | 78C > T; | | | L213P; | | | al, 1999 |
| | | 1979T > C; | | | R296C; | | | |
| | | 2850C > T; | | | S486T | | | |
| | | 4180G > C | | | | | | |
| CYP2D6*21 | CYP2D6.21 | 77G > A | | M1 | R26H | | | Marez et |
| | | | | | | | | al, 1997 |
| CYP2D6*22 | CYP2D6.22 | 82C > T | | M2 | R28C | | | Marez et |
| | | | | | | | | al, 1997 |
| CYP2D6*23 | CYP2D6.23 | 957C > T | | M3 | A85V | | | Marez et |
| | | | | | | | | al, 1997 |
| CYP2D6*24 | CYP2D6.24 | 2853A > C | | M6 | I297L | | | Marez et |
| | | | | | | | | al, 1997 |
| CYP2D6*25 | CYP2D6.25 | 3198C > G | | M7 | R343G | | | Marez et |
| | | | | | | | | al, 1997 |
| CYP2D6*26 | CYP2D6.26 | 3277T > C | | M8 | I369T | | | Marez et |
| | | | | | | | | al, 1997 |
| CYP2D6*27 | CYP2D6.27 | 3853G > A | | M9 | E410K | | | Marez et |
| | | | | | | | | al, 1997 |
| CYP2D6*28 | CYP2D6.28 | 19G > A; | | M11 | V7M; | | | Marez et |
| | | 1661G > C; | | | Q151E; | | | al, 1997 |
| | | 1704C > G; | | | R296C; | | | |
| | | 2850C > T; | | | S486T | | | |
| | | 4180G > C | | | | | | |
| CYP2D6*29 | CYP2D6.29 | 1659G > A; | | M13 | V136M; | | | Marez et |
| | | 1661G > C; | | | R296C; | | | al, 1997 |
| | | 2850C > T; | | | V338M; | | | |
| | | 3183G > A; | | | S486T | | | |
| | | 4180G > C | | | | | | |
| CYP2D6*30 | CYP2D6.30 | 1661G > C; | | M15 | 172- | | | Marez et |
| | | 1855-1863 | | | 174FRP | | | al, 1997 |
| | | 9bp rep; | | | rep; | | | |
| | | 2850C > T; | | | R296C; | | | |
| | | 4180G > C | | | S486T | | | |
| CYP2D6*31 | CYP2D6.31 | 1661G > C; | | M20 | R296C; | | | Marez et |
| | | 2850C >0 T; | | | R440H; | | | al, 1997 |
| | | 4042G > A; | | | S486T | | | |
| | | 4180G > C | | | | | | |
| CYP2D6*32 | CYP2D6.32 | 1661G > C; | | M19 | R296C; | | | Marez et |
| | | 2850C > T; | | | E410K; | | | al, 1997 |
| | | 3853G > A; | | | S486T | | | |
| | | 4180G > C | | | | | | |
| CYP2D6*33 | CYP2D6.33 | 2483G > T | | CYP2 | A237S | Normal | | Marez et |
| | | | | D6*1C | | (s) | | al, 1997 |
| CYP2D6*34 | CYP2D6.34 | 2850C > T | | CYP2 | R296C | | | Marez et |
| | | | | D6*1D | | | | al, 1997 |
| CYP2D6*35 | CYP2D6.35 | 31G > A; | | CYP2 | V11M; | Normal | | Marez et |
| | | 1661GC; | | D6*2B | R296C; | (s) | | al, 1997 |
| | | 2850G > T; | | | S486T | | | |
| | | 4180G > C | | | | | | |
| CYP2D6*35X2 | CYP2D6.35 | 31G > A; | | | V11M; | Incr | | Griese et |
| | | 1661G > C; | | | R296C; | | | al, 1998 |
| | | 2850C > T; | | | S486T | | | |
| | | 4180G > C | | | | | | |
| CYP2D6*36 | CYP2D6.36 | 100C > T; | 44,29 | CYP2 | P34S; | Decr | Decr | Wang, |
| | | 1039C > T; | | D6Ch2 | S486T | (d) | (b) | 1992 |
| | | 1661G > C; | | | | | | Johansson |
| | | 4180G > C; | | | | | | et al, 1994 |
| | | gene | | | | | | Leathart et |
| | | conversion to | | | | | | al, 1998 |
| | | CYP2D7 in | | | | | | |
| | | exon 9 | | | | | | |
| CYP2D6*37 | CYP2D6.37 | 100C > T; | | CYP2 | P34S; | | | Marez et |
| | | 1039C > T; | | D6*10D | S486T; | | | al, 1997 |
| | | 1661G > C; | | | R201H; | | | |
| | | 1943G > A; | | | S486T | | | |
| | | 4180G > C; | | | | | | |
| CYP2D6*38 | | 2587-2590 | | N2 | Frame | None | | Leathart et |
| | | delGACT | | | shift | | | al, 1998 |

b, bufuralol; d, debrisoquine; dx, dextromethorphan; s, sparteine

References from above table:

Aklillu et al., J. Pharmacol. Exp. Ther., (1996), 278:441-446.

Broly, F. and Meyer, U. A., Pharmacogenetics, (1993), 3:123-130.

Broly, F., et al., Pharmacogenetics, (1995), 5:373-384.

Broly et al., Hum. Genet., (1995), 96:601-603.

Dahl, M., et al., J. Pharmacol. Exp., (1995), 274:516-520.

Daly, A. K., et al., Human Genet., (1995), 95:337-341.

Daly et al., Pharmacogenetics, (1996), 6:319-328.

Evert et al., Pharmacogenetics, (1994), 4:271-274.

Evert, B., et al., Naunyn-Schmiedeberg's Arch. Pharmacol., (1994), 350:434-439.

Gaedigk, A., et al., Am. J. Hum. Genet., (1991), 48:943-950.

Gough, A. C., et al., Nature, (1990), 347:773-776.

Griese et al., Pharmacogenetics, (1998), 8:15-26.

Hanioka, N., et al., Am. J. Hum. Genet., (1990), 47:994-1001.

Johansson, I., et al., Proc. Natl. Acad. Sci., USA, (1993), 90:11825-11829.

Johanson, I., et al., Mol. Pharmacol., (1994), 46:452-459.

Kagimoto, M., J. Biol. Chem., (1990), 265:17209-17214.

Kimura, S., et al., Am. J. Hum. Genet., (1989), 45:889-905.

Leathart et al., Pharmacogenetics, (1998), 8:529-541.

Lovlie et al., Pharmacogenetics, (1997), 7:149-152.

Marez, D., et al., Pharmacogenetics, (1995), 5:305-311.

Marez et al., Hum. Genet., (1996), 97:668-670.

Marez et al., Pharmacogenetics, (1997), 7:193-202.

Marez-Allorge et al., Pharmacogenetics, (1999), 9:393-396.

Masimirembwa et al., Br. J. Clin. Pharmacol., (1996), 42:713-719.

Oscarsen et al., Mol. Pharmacol. (1997), 52:1034-1040.

Panserat, S., et al., Hum. Genet., (1994), 94:401-406.

Panserat, S., Br. J. Clin. Pharmacol., (1995), 40:361-367.

Sachse, C., et al., Pharmacogenetics, (1996), 6:269-272.

Sachse et al., Am. J. Hum. Genet., (1997), 60:265-271.

Sachse et al., Pharmacogenetics (1998) 8(2):181-185.

Saxena, R., et al., Hum. Mol. Genet., (1994), 3:923-926.

Steen, V. M., et al., Hum. Mol. Genet., (1995), 4:2251-2257.

Tyndale, R., et al., Pharmacogenetics, (1991), 1:26-32.

Wang, S., Master's Thesis, Nat'l Cheng Kung University, Tainan, Taiwan (1992).

Wang et al., Drug Metab. Dispos., (1999), 27:385-388.

Yokoi et al, Pharmacogenetics, (1996), 6:395-401.

Yokota, H., et al., Pharmacogenetics, (1993), 3:256-263.

An exemplary embodiment of the present invention designated, the CYP2D6 Mutation Detection System (MDS-CYP2D6), utilizes Polymerase Chain Reaction (PCR), Resonance Light Scattering (RLS) particles, and nucleic acid hybridization technology to analyze the genotype of five specific alleles of the Cytochrome P450 2D6 (CYP2D6) gene as described in the Examples.

Following is a set of references providing information on CYP2D6, and on pharmacogenetics associated with that gene.

Bertilsson L, Lou YQ, Du YL, Liu Y, Kuang TY, Liao XM, et al. Pronounced differences between native Chinese and Swedish populations in the polymorphic hydroxylations of debrisoquin and S-mephenytoin. Clin Pharmacol Ther 1992;51:388-97 [Published erratum appears in Clin Pharmacol Ther 1994;55:648].

Cholerton S, Daly AK, Idle JR. The role of individual human cytochromes P450 in drug metabolism and clinical response. Trends Phannacol Sci 1992;13: 434-9.

Crewe HK, Lennard MS, Tucker GT, Woods FR, Haddock RE. The effect of selective serotonin re-uptake inhibitors on cytochrome P4502D6 (CYP2D6) activity in human liver microsomes. Br J Clin Pharmacol 1992;34:262-5.

Daly AK, Brockmiller J., Broly F., et al. Nomenclature for Human CYP2D6 Alleles. Pharmacogenetics 1996;6: 193-201

Hamelin BA, Dorson PG, Pabis D, Still D, Bouchard RH, Pourcher E, Rail J, Turgeon J, Crismon ML. CYP2D6 mutations and therapeutic outcome in schizophrenic patients. Pharmacotherapy 1999 Sep;19(9): 1057-63

Knodell RG, Browne DG, Gwozdz GP, Brian WR, Guengerich FP. Differential inhibition of individual human liver cytochromes P-450 by cimetidine. Gastroenterology 1991;101:1680-91.

Philip PA, James CA, Rogers HJ. The influence of cimetidine on debrisoquine 4-hydroxylation in extensive metabolizers. Eur J Clin Pharmacol 1989;36:319-21.

Phillips, IR. and Shephard, EA (eds), Cytochrome P450 Protocols, Volume 107, Humana Press, 1998.

Pollock BG, Mulsant BH, Sweet RA, Rosen J, Altieri LP, Perel JM. Prospective cytochrome P450 phenotyping for neuroleptic treatment in dementia. Psychopharmacol Bull 1995;31:327-31.

Relling MV, Cherrie J, Schell MJ, Petros WP, Meyer WH, Evans WE. Lower prevalence of the debrisoquin oxidative poor metabolizer phenotype in American black versus white subjects. Clin Pharmacol Ther 1991;50:308-13.

Richardson, JH. and W. Emmett Barkley (eds), “Biosafety in Microbiological and Biomedical Laboratories.” HHS Publication Number [CDC]88-8395.

Steiner E, Bertilsson L, Sawe J, Bertling I, Sjoqvist F. Polymorphic debrisoquin hydroxylation in 757 Swedish subjects. Clin Pharmacol Ther 1988; 44:431-5.

Spina E, Ancione M, Di Rosa AE, Meduri M, Caputi AP. Polymorphic debrisoquine oxidation and acute neuroleptic-induced adverse effects. Eur J Clin Phannacol 1992;42:347-8.

Tollefson GD. Adverse drug reactions/interactions in maintenance therapy. J Clin Psych 1993;54 (Suppl):48-60.

Van der Weide J, Steijns LS. Cytochrome P450 enzyme system: genetic polymorphisms and impact on clinical pharmacology. Ann Clin Biochem 1999 Nov;36 (Pt 6): 722-9

Yue QY, Svensson JO, Alm C, Sjoqvist F, Sawe J. Codeine O-demethylation co-segregates with polymorphic debrisoquine hydroxylation. Br J Clin Pharmacol 1989;28:639-45.

C. Exemplary Assay Formats

As will be recognized by those skilled in the art, assays can be constructed in many different formats. Thus, assays can be carried out singly, but preferably for allele identification, the assay is carried out as an integrated set of assays. Preferably the set characterizes the presence or absence of particular mutant or wild type sequences at particular sites in a gene. Examples of formats allowing convenient determination of polymorphisms at a plurality of sites include microtiter plates, arrays slides, array chips, and other multi-spot or multi-well formats. Such formats typically utilize glass, plastic, filters, or membranes as solid supports. Spots can be of various sizes, e.g., less than 1 μm², 1-10 μm², 10-100 μm², 100-1000 μm², 0.01-0.1 mm², 0.1-1.0 mm², 1-10 mμm², and 10-50 mm², or even larger.

Preparation of arrays on slides or other surfaces, is well-known in the art. A number of different methods for preparing slides and depositing spots are described, for example, in Microarray Biochip Technology, Mark Schena, ed., for example, in Chapters 2 and 3 and in references cited therein. Methods include, for example, pin spotting, piezoelectric deposition, ink jet technology, and hand spotting. Those skilled in the art understand how to select appropriate deposition methods and conditions depending, for example, on the materials to be deposited, number and size of spots, number of slides, and consistency requirements.

In construction of solid phase assays for the present invention based on nucleic acid hybridization, generally one of two configurations will be used. In the first, capture probes are immobilized to a solid phase. Labeled target representing the genomic region containing the SNP is hybridized. In this configuration, capture probes are preferably designed to create the greatest differential hybridization between the capture probe and the homologous and SNP-containing target. After hybridization and stringent washing to impart differential hybridization as above, RLS particles are bound to the captured targets, and the bound target molecules are detected by detecting light scattering from the RLS particles.

The second configuration is similar to the above configuration, except that captured target is not directly labeled. Rather, a labeled detection probe is hybridized to the captured target before, during, or subsequent to target hybridization to the immobilized capture probe. SNP specificity can be designed into either the immobilized capture probe or the labeled detection probe or both.

D. Target Nucleic Acid Preparation

Target nucleic acid can be prepared for a target sequence assay by a number of different methods, some of which involve amplification and some of which do not. The exemplary method described in the examples utilizes PCR.

In addition, the target nucleic acid may be labeled by incorporation of a moiety that provides attachment for additional molecules, particles, or moieties, and/or provides useful properties such as providing a cleavage site. An example is the incorporation of biotinylated nucleotides to provide binding of anti-biotin antibodies or avidin/streptavidin. Another example is the incorporation of bromodeoxyuridin (BrdU). BrdU provides both a cleavage site and an attachment site, e.g., using anti-BrdU antibodies.

As indicated, an exemplary system utilizes PCR, and preferably involves amplification and incorporation of biotinylated nucleotides.

Non-PCR Incorporation Labeling

Given the sensitivity of the RLS particle signal generation and detection technology on developed simple array systems, a sufficient number of copies of a single copy human gene is present in a reasonable volume of human blood for detection without the need for PCR. For example, genomic DNA was recently prepared from blood using a commercially available kit to obtain a yield of 20 μg/ml. This amount of human DNA corresponds to approximately 7×10⁶ copies of a single copy gene. Thus from 10 ml of blood, approximately 7×10⁷ copies can be obtained. This indicates one can detect single copy genes in human DNA without PCR by primer extension through the target region of interest with incorporation of either a hapten or modified base (see below) that is subsequently detected by specific antibodies on RLS particles or other specific binding interaction.

Incorporation Labeling of Genes in Human Genomic DNA

Three basic, exemplary, non-limiting approaches for incorporation labeling will be initially pointed out:

1. Random-prime labeling using randomshort, e.g., hexamer, primers, Klenow fragment of DNA polymerase I at 37° C.

2. “Biased” random-prime labeling using random flanking “gene region-specific” fragments as primers and a DNA polymerase, such as Bst DNA polymerase at 60° C.

3, Primer extension using a mixture of two or more opposing gene-specific primers adjacent to the region of interest and a DNA polymerase, such as Bst DNA polymerase at 60° C.

All three methods will provide for at least some level of target amplification by strand displacement. This means that lower volume (i.e. <10 ml) of blood may be used for the system.

Method 1 is applicable, for example, to potential micorarray haplotyping applications (i.e. the simultaneous genotyping of numerous genes/SNPs). Method 1 is preferably not applied to genes such as CYP2D6, where there are closely related (and cross-hybridizing) members of the gene family, e.g., the cytochrome P450 gene family and pseudogenes also present in human DNA.

Method 2 primers can, for example, be prepared by DNAse digestion of PCR products that are adjacent to the region of interest. This method can provide a measure of region-specific extension and considerable strand displacement. Large amounts of opposing region-specific primer for this method of incorporation labeling can be prepared from appropriate flanking fragments cloned in vectors, e.g., phagemid vectors.

Method 3 can provide better specificity of primer extension through the region of interest, although with a lower level of target amplification due to strand displacement.

PCR-Free Incorporation Labeling for Array-Based SNP Detection

Common to the various methods for non-PCR incorporation labeling is polymerase extension through the target region of interest with enzymatic incorporation of a base entity that can be subsequently detected by appropriately derivatized RLS particles. Polymerase extension can be either gene-specific or more general according to the application and the number of genomic target sequences to be detected. For RLS detection of hybridized genomic targets, a variety of derivatized (e.g. biotin) or modified DNA bases (e.g. BrdU) for which specific antibodies or other specific binding moieties can be included in extension reactions as described below. Preferably the incorporation labeling system includes:

preparation of genomic DNA from blood

general or locus specific incorporation labeling

processing and/or hybridization of the labeled target DNA to an array

detection of the hybridized target sequence(s) using RLS particles

Other desired characteristics for the system are

a minimum input mass of human genomic DNA (0.1 to 5 μg)

a minimum number of steps

preferably one but no more than 2 different enzymatic components (e.g. polymerases).

An exemplary systems utilizes biotin incorporation, but other systems utilize alternative haptens or modified bases that can be detected by appropriately derivatized RLS particles.

Incorporation Labeling Approaches

Several techniques for incorporation labeling can be utilized, each with different preferred uses and applications. For genetic analysis for a large number of dispersed SNPs, several general incorporation labeling strategies can be used. For detection of a relatively small number of known SNPs, several methods can be utilized to target incorporation labeling specifically in the genomic region(s) of interest (e.g. in the CYP2D6 gene). Each approach and its primary use in SNP applications is described below.

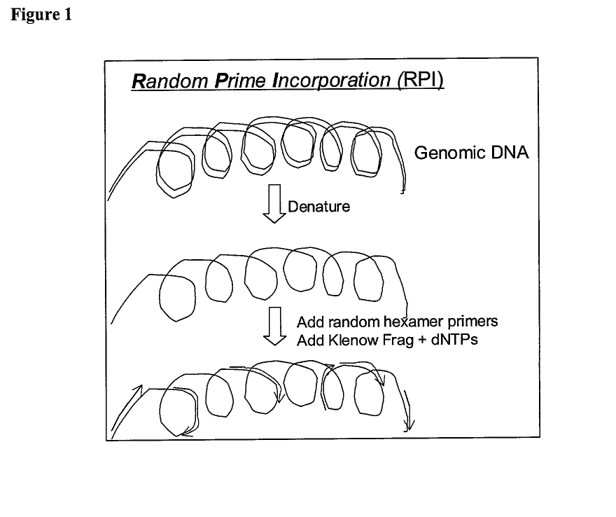

1. Approach: Random-prime labeling (Bert and Vogelstein, 1984) using random hexamer primers and Klenow fragment of DNA polymerase I. Preferred application is for the general incorporation labeling of genomic DNA target sequences for RLS detection of a large number of SNPs. This method is shown schematically in FIG. 1

In this approach, genomic DNA is isolated, denatured and treated with the Klenow fragment of DNA polymerase I in the presence of random “hexamer” primers and dNTPs. The primers in this system hybridize throughout the genomic DNA in a largely random fashion. These in turn are extended by the Klenow enzyme, which lacks 3′-5′ exonuclease proofreading activity, in the 5′-3′ direction. This enzyme is also capable of affecting strand displacement with good efficiency, thus a modest level of target amplification is afforded by this mechanism.

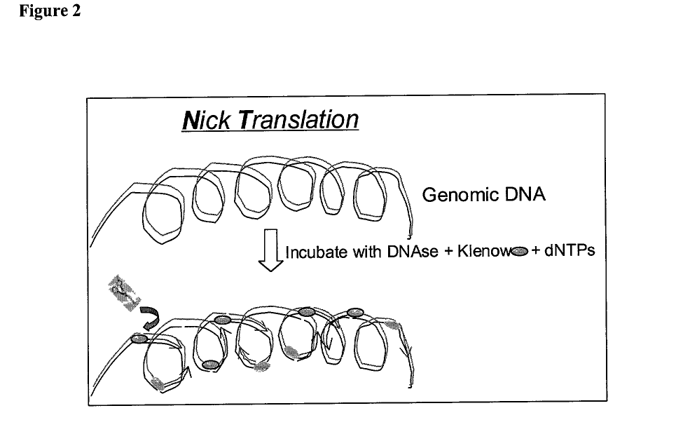

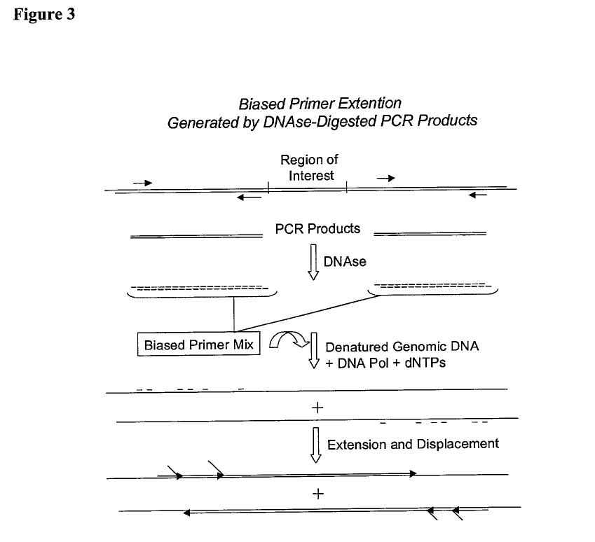

2. Approach: Nick-Translation of total human genomic DNA. A preferred application is for the general incorporation labeling of genomic DNA target sequences for RLS detection of a large number of SNPs. The approach is shown schematically in FIG. 2.

In nick translation incorporation labeling of genomic DNA, double stranded genomic DNA is nicked using trace amounts of DNAse I to generate single-stranded nicks bearing free, 3′ hydroxyl groups that function as initiation sites for extension by DNA polymerases. Nicks in the genomic DNA have a quasi-random distribution and can be introduced either in a step preceding or during the extension reaction. Typically, the Klenow fragment of DNA polymerase I is used to catalyze the incorporation of dNTPs in the extension reaction. Trace amounts of DNAse 1 in the reaction perpetuates the reaction cycle and some level of target amplification occurs via strand displacement by the Klenow enzyme. Nick translation labeling of DNA is described, for example, in Rigby et al, 1977. Nick translation has been used for incorporation labeling of genomic DNA to generate fluorescent genomic DNA target that is used for a BAC array-based cancer screen that detects gene amplification at a large number of loci (David Lane, Vysis, ATP National Meeting, 1999).

3. Approach: “Biased” random-prime labeling using random flanking “gene region-specific” fragments as primers and a thermostable DNA polymerase. A preferred application is for incorporation labeling of one or more target gene regions of interest. The technique is shown schematically in FIG. 3.

This approach is suited, for example, for incorporation labeling of one or more target gene regions of interest. In this method, one initially generates a population of “biased primers” by randomly digesting specific PCR products representing genomic regions that immediately flank the target region of interest. Genomic DNA is prepared, denatured and annealed to primers generated in this manner in the presence of dNTPs and a thermostable DNA polymerase. Primers prepared in this manner will range in size and preferentially hybridize at an appropriate restrictive temperature to the flanking regions where the DNA polymerase will extend them through the adjacent target region of interest. It should be noted that whereas this method of primer preparation is the simplest for early development, the primers are functionally bi-directional. This indicates that sensitivity and specificity in the developed prototype system can be substantially improved by preparing primers from the appropriate single strand templates (i.e. the same flanking PCR regions excised from single stranded phagemid constructs). This approach will provide incorporation labeling that is more region-specific in comparison to the general labeling methods above. In addition, this approach will provide a significant level of target amplification via strand displacement. A variety of thermostable DNA polymerases can be used with this method.

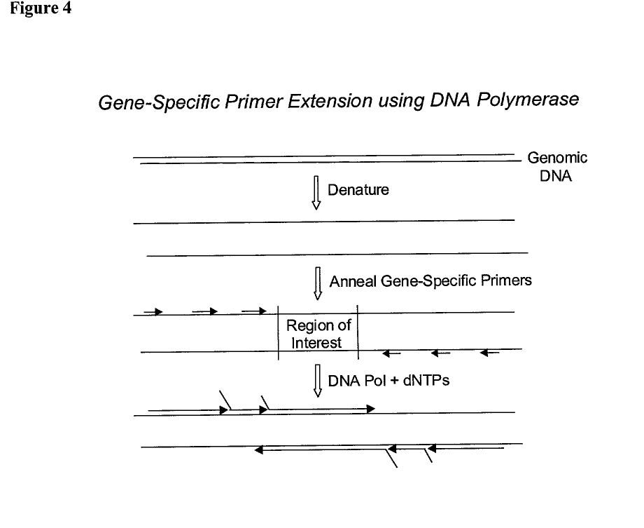

4. Approach: Primer extension using a mixture of two or more opposing gene-specific primers adjacent to the region of interest and a thermostable DNA polymerase. A preferred application is for gene-specific incorporation labeling. The technique is shown schematically in FIG. 4.

This approach is well-suited for RLS detection of a modest number of SNP loci that may be broadly distributed throughout the genome. In this method, one first designs a series of gene-specific primers, each of which hybridizes to a particular target sequence that flanks the SNP region of interest. Prepared genomic DNA is denatured and incubated with the primers, dNTPs and a thermostable DNA polymerase at an optimized operating temperature. During the incubation, the primers hybridize and the thermostable DNA polymerase extends the primers in the appropriate direction through the region of interest. In this manner, labeled dNTPs are incorporated into the genomic target sequence of interest.

Specificity of the primers is important in this method, and this property can be imparted by careful primer design including the creation of internal destabilizing and 3′-end mismatches with other closely related sequences (e.g. in homologous genes within a gene family) present in human genomic DNA. Some level of target amplification can be obtained by strand displacement in this method by including multiple primers for a given region of interest. Primer ratios should be stoichiometrically and kinetically optimized for a given primer set for efficient strand displacement. This approach utilizes techniques as in primer extension mapping, an established technique used in gene mapping and structure analysis.

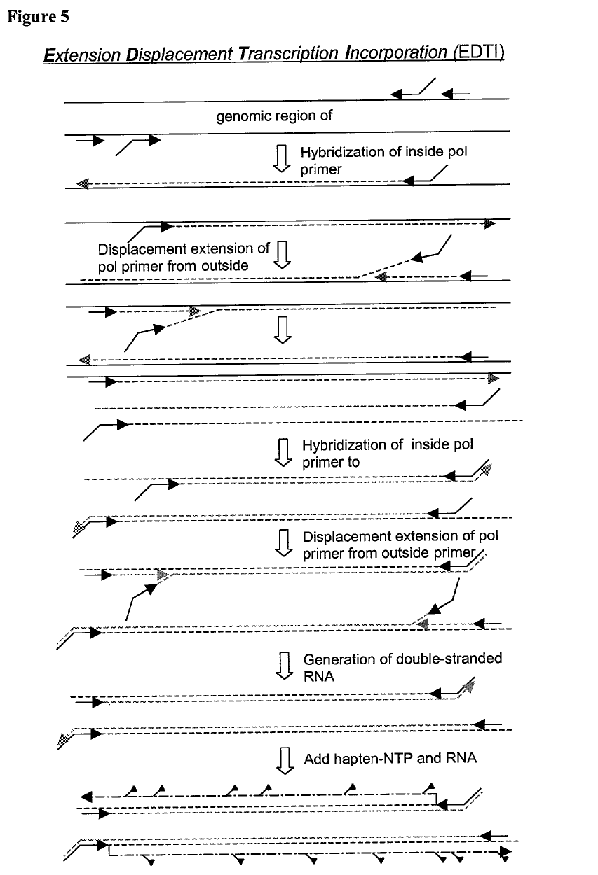

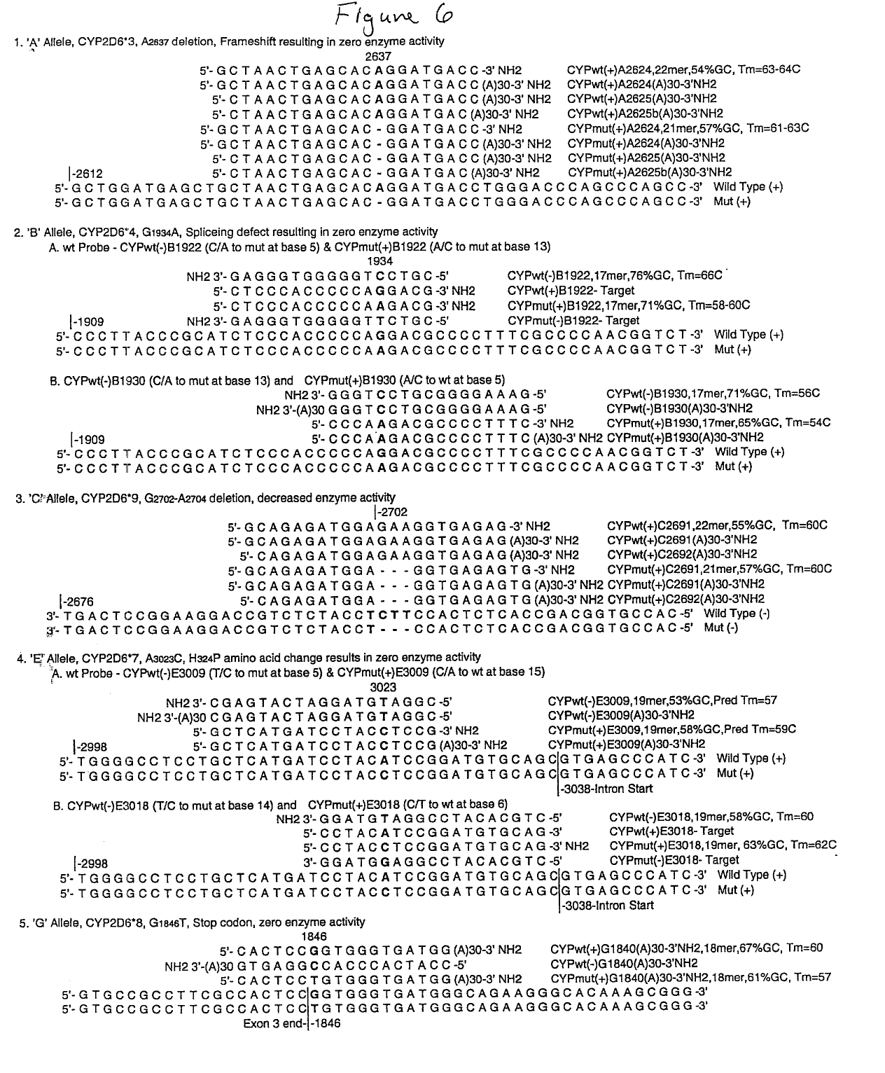

5. Approach: Extension Displacement Transcription Incorporation (EDTI). A preferred application is in gene-specific incorporation labeling. The technique is shown schematically in FIG. 5.

Incorporation labeling by EDTI proceeds as a two-step process and has a significant degree of target amplification. In the first step of the process, the genomic DNA template is denatured and hybridized with multiple, opposing gene-specific primers in the presence of a thermal stable DNA polymerase and dNTPs to generate and displace extension products through the region of interest. As with the procedure described above for primer extension incorporation labeling, primer design, primer ratio and optimization of the system at a given operating temperature is important for the specificity of the extension reaction and the efficiency of strand displacement. In this first step, extension of directionally opposing primers and displacement activities combine to generates a double stranded template that is used in the second step in for the in vitro transcription reaction. Thus, following the first step comprising primer extension and displacement, reaction conditions (buffers and temperature) are adjusted and an RNA polymerase and NTPs are added. This step affords at least 100-fold target amplification with incorporation of a labeled base in sequences representing the target region of interest. The labeled RNA products are then hybridized to the array for SNP detection. A related method has been described for preparation of targets for RNA expression analysis (see below, Van Gelder et al, 1999).

All of the incorporation labeling approaches described above require a high level of sensitivity; in cases that target a more limited set of specific gene targets, specificity of the incorporation labeling process is also required. It has been found that the sensitivity of RLS particle detection is similar to the sensitivity obtained using ³²p incorporation and phosphorimaging. As such, it has been recognized that incorporation labeling of nucleic acid targets with radioisotopes represents an approach that affords the greatest sensitivity in many applications.

One skilled in the art will recognized that the incorporation labeling methods detailed above are non-limiting in the present invention. Other methods for obtaining various levels of SNP-containing target amplification and incorporation labeling, either using labeled nucleoside triphosphates or internally or terminally labeled primers can be employed. Non-limiting examples of other target amplification or labeling methods include ligase chain reaction (U.S. Pat. No, 5,516,663, U.S. Pat. No. 5686272, U.S. Pat. No. 5,869,252, U.S. Pat. No. 6,143,527), ligation of multiple oligomers amplification (U.S. Pat. No. 5,998,175, U.S. 6,001,614, U.S. 6,013,456, U.S. 6,020,138), rolling circle amplification (U.S. Pat. No. 6,221,603), strand displacment amplification (Walker et al. 1993 and Walker 1995), transcription mediated amplification (Kacian et al. 1996 and Cleuziat et al. 1998), and the like.

Biotin Alternatives

A variety of alternatives to biotin including other haptens derivatized to nucleoside triphosphates, such as fluorescein, digoxigenin and dinitrophenol, exist, and can be used for incorporation labeling and detection with RLS particles. In addition one can incorporate a specific functional group into the desired target nucleic acid using, for example, allyl-amino dUTP, to which a variety of haptens or other labels can be chemically coupled. One alternative is incorporation of bromodeoxyuridine (BrdU), a base analog of thymidine. This system features several potential advantages over biotin/anti-biotin and other hapten/anti-hapten RLS systems including reduced reagent cost, increased incorporation efficiency, and beneficial chemical properties for post incorporation labeling processing. Several sources of anti-BrdU antibodies are available for derivatization of RLS particles and previous results have demonstrated anti-BrdU RLS particle detection on array slides. One skilled in the art will recognize that other base analogs for which antibodies are available can also be useful for optimizing alternative procedures for the substitution of the biotin-antibiotin or streptavidin system.

The following patents and publications provide usefultechniques that can be utilized in embodiments of the present invention.

Strand displacement amplification (SDA): Walker et al. 1993 and Walker 1995 describe this target amplification approach that uses hybridization of a specific primer to generate a hemi-methylated restriction site and a restriction endonuclease to generate a specific proximal nick which is extended through the region of interest by a DNA polymerase.

Random primer extension: Hartley and Berninger, 1992, describe random primer labeling. Similar methods are described in U.S. Pat. No. ,5106,727, which were described in Bert and Vogelstein, 1984

Extension/Transcription Systems: Kacian et al. 1996 and Cleuziat et al. 1998 describe target amplification approaches known as TMA and NASBA. Both systems use reverse transcriptase and RNAse H activities, neither of which is associated with the approaches for incorporation labeling discussed above. Van Gelder et al. 1999 describes methods for amplification of RNA sequence in expression studies. This approach requires the initial extension reaction to be carried out on an RNA strand by reverse transcriptase.

Haptens: Haptenylated dNTP or NTPs for incorporation labeling in some cases have been described. For example, the Ward et al. patents and patents for digoxigenin incorporation labeling/detection, and Huber et al. describe a number of haptens.

### REFERENCES

Cleuziat et al. (1998) Method for nucleic acid amplification by transcription using displacement, and reagents and kit therefor. U.S. Pat. No. 5,849,547.

Feinberg and Vogelstein (1984) Anal Biochem. 137, 266-267.

Hartley and Berninger (1992) Amplification of nucleic acid sequences using oligonucleotides of random sequences as primers. U.S. Pat. No. 5,106,727.

Huber et al. (1993) Digoxigenin derivatives and use thereof. U.S. Pat. No. 5,198,537.

Kacian et al. (1996) Nucleic acid sequence amplification methods. U.S. Pat. No. 5,480,784.

Rigby PW et al. (1977) J Mol Biol 113(1), 237-51.

Van Gelder et al. (1999) Processes for genetic manipulations using promoters. U.S. Pat. No. 5,891,636.

Walker et al. (1993) Nucleic acid target generation. U.S. Pat. No. 5,270,184.

Walker (1995) Strand Displacement Amplification. U.S. Pat. No. 5,455,166.

Ward et al. (1987) Modified nucleotides and methods of preparing and using same. U.S. Pat. No. 4,711,955.

Ward et al. (1995) Modified nucleotides and methods of preparing and using same. U.S. Pat. No. 5,449,767.

Incorporation Labeling of DNA Targets with Bromodeoxyuridine (BrdU) and RLS Detection

Incorporation of the base analog 5-BrdU into DNA has been an established method for mutagenesis, in DNA replication studies, and analysis of apoptosis. In these approaches, cells in culture are treated with BrdU under various conditions. This modified base is converted to the deoxyribose triphosphate intracellularly prior to incorporation of BrdU during DNA synthesis. Recently, monoclonal antibody reagents specific for BrdU have become commercially available to detect BrdU incorporated in vivo in nuclear DNA for studying apoptosis. In vitro, many DNA polymerases and reverse transcriptases used for nucleic acid labeling incorporate BrdU with high efficiency. In addition, methods for controlled fragmentation of BrdU incorporated DNA have been described in the literature, and this process can be modulated by a variety of experimental conditions.

The cost of BrdU incorporation is substantially less than for other bases modified with various haptens (e.g. biotin or dig-dNTPs). BrdU can be incorporated both enzymatically for target labeling and during automated DNA synthesis, e.g., primer synthesis. Experiments that demonstrate incorporation labeling and RLS detection of BrdU DNA targets are briefly described below.

Incorporation Labeling with BrdU and Fragmentation

Exemplary detection methods described herein for cDNA or PCR amplicons has utilized incorporation of biotin-dUTP. This is expensive and incorporation efficiencies are variable depending on the system. 5-bromodeoxyuridine is incorporated with high efficiency by DNA polymerases and reverse transcriptase. High affinity monoclonal and affinity purified polyclonal antibodies are available for specific detection of BrdU incorporated DNA. BrdU incorporated DNA can also be cleaved in a controllable manner by a variety of treatments including base, heat and UV light. Cleavage of target prior to hybridization may be important for efficient hybridization and detection.

In summary, BUdR (or another modified base) incorporation is expected to be substantially less expensive on a per reaction or sample basis, more efficient and provide a DNA target that can be controllably cleaved and specifically detected.

Incorporation of BUd

BrdU can be incorporated during PCR using appropriate enzymes, such as Taq polymerase. Typically PCR is performed on genomic DNA template for 30 cycles or less using various CYP2D6 gene-specific primers or, in other systems, primers appropriate for the particular target gene region. Incorporation efficiency, as measured by the relative amount of specific PCR product generated, was compared with incorporation of dUTP-biotin at various levels. Products generated with 20% BrdU showed approximately the same level as for biotin-containing reactions and for unmodified

In many cases, fragmentation of labeled DNA products is beneficial or necessary for efficient hybridization to capture probes on microarray surfaces. Typically, this step is difficult to control and relies upon DNAse 1 treatment or other enzymatic process. Fragmentation can also be accomplished using cleavage at BrdU analogs with BrdU incorporation at appropriate levels. Those skilled in the art will readily be able to determine the incorporation level to generate appropriate length fragments. Cleavage can be performed using any of a variety of treatments, e.g., treatment by incubation in a heat block 95° C. for 120 minutes. Fragments in the range of several hundred bases in length appear to hybridize the most efficiently and be detected by RLS particles under typical experimental conditions. Heat cleavage of BrdU incorporated DNA is more efficient on single stranded DNA (therefore heating IPCR product at 95° C.) and upon cleavage there is roughly an equal chance that a given BrdU base will remain on the 5′ or 3′ end of the cleavage products. Experiments have indicated that the BrdU cleavage process can be accelerated by altering the pH and salt conditions.

While not limited to this use, BrdU-labeled DNA can be used in detection methods utilizing microarrays. For example, to examine RLS detection of BrdU-labeled DNA targets on microarrays, an anti-BrdU monoclonal antibody was employed. As for other antibody systems, salt conditions for optimal adsorption onto RLS particles were determined empirically a priori. Once these conditions were established, 80 nm gold RLS particles coated with the anti-BrdU antibody were prepared. These particles were tested in a model system and used in experiments for detection of BrdU-labeled and processes PCR products.

Results of a sensitivity dilution experiment where a synthetic oligonucleotide at 10 μM with a single 5′ BrdU and 52 bases long representing the CYP2D6 “A” allele was spotted onto a carboxylated glass slide with dilution using the unlabeled oligonucleotide of the same sequence demonstrated that one can see good sensitivity (down to 1/1000 dilution) with this system.

Anti-BrdU RLS particles were also used to detect BrdU-incorporated and processed PCR products in the CYP2D6 microarray assay. BrdU-incorporated PCR product was generated using defined conditions (50% BrdU:50% dTTP) in a CYP2D6 multiplex PCR reaction. For comparison, a parallel reaction was run using a 30/70 ratio of biotin-16 dUTP:dTTP. 10 μl of both reactions were processed and hybridized to CYP2D6 hand-spotted microarrays containing allele-specific capture probes. After washing, the arrays were blocked and reacted with either anti-BrdU or anti-biotin RLS particles.

While not as extensively developed as the previously optimized biotin incorporation-anti-biotin RLS particle system, strong signals with equivalent specificity were obtained in this experiment. These results clearly demonstrated the utility of this method for incorporation labeling and RLS particle detection.

In addition to the incorporation labeling and strand displacement methods described above, additional sample preparation methods can be utilized. The simplest involves no DNA synthesis, but rather utilizes digestion, with an allele enrichment method. For example, the enrichment can be provided by capture with an allele-specific capture probe. In this method, appropriate selection of restriction enzymes can produce nucleic acid fragments of appropriate size for use in the present detection methods. Indeed, in some cases, a cleavage site can be selected that includes a polymorphic site, so that one allelic form will be cleaved and the other will not. Then, a simple capture probe used in conjunction with size separation can provide a high level of allele enrichment.

Thus, included in this invention are multiple methods for sample preparation. These include PCR amplification of a nucleic acid sequence, generally including a target sequence.

D. Light Scattering Particles

Preferably an assay, e.g., a the CYP2D6 mutation assay, is performed using Resonance Light (RLS) Scattering Particles. Preferred RLS particles are composed of colloidal metals, preferably gold, silver, mixed gold and silver, or other mixed composition particles containing gold and/or silver. A large number of methods for preparing gold or silver colloids have been described. Examples are provided in the references below and in the Yguerabide et al. references cited in the Summary, along with methods for attaching such particles to other molecules for attachment to a binding or targeting moiety.

Those skilled in the art are familiar with a number of different methods for preparing gold or silver particles. For example, typically such gold particles, are formed by reducing gold chloride with various reducing agents (depending on desired particle size) such as white phosphorous, tannic acid, and sodium citrate. For a review on the synthesis of colloidal gold particles see Horisberger, Jennes, and Frens, cited below.

Colloidal gold particles have a net negatively charged surface and can be coated and stabilized using biological molecules. The process of adsorption, which is a non-covalent binding, is caused by coulomb forces, electrostatic interaction, and by van der Waal forces and depends on different factors such as pH, ionic strength, concentration, temperature, or electrolytes. For a review on protein adsorption on colloidal gold see Jennes, Geoghegan, Molina-Bolivar, Ramano and Leuvering cited below.

### REFERENCES

Horisberger, M. (1981), Scan Electron Microsc., v2, p9-28; and Trends Biochem. Sci. (1983), v7, p395-397.

Jennes, L. et al (1986), Methods in Enzymology, v124, p36-47.

Frens, G. (1973), Nature Phys. Sci., v241,p20-22.

Geoghegan, W. et al (1977), J. Histochem. Cytochem., v25, p1 187-1200.

Molina-Bolivar, J. A. et al, (1999), Langmuir, v15, p2644-2653.

Ramano, E. L. et al (1974), Immunohistochmistry, v11,p521-522.

Leuvering, (Feb. 2, 1982) U.S. Pat. No. 4,313,734.

E. Probe-Particle Attachment and Capture Probe Solid Phase Substrate Attachment

For use in the present invention, a slide or other solid phase device, e.g., a glass slide is preferably surface treated or coated. Examples of such treatment is treatment with casein, functionized silane compounds, or polymer coating including polylysine or a polymer matrix.

Once coated, a particle can be attached to a biomolecule or other convenient molecule using conventional chemistries. The appropriate chemistry to use will be apparent to those skilled in the art, depending on the available functional groups and the chemical characteristics of the molecule to be attached.

Various methods have been developed to analyze nucleic acid molecules present in experimental or diagnostic samples. Many of these techniques are assays wherein the sample is placed in contact with a solid support. The solid support contains nucleic acid molecules which have been immobilized by covalent or noncovalent attachment. Immobilization of a nucleic acid molecule to a spatially defined position on a solid support can be used in many ways. These uses include: hybridization assays which are able to identify an individual nucleic acid of interest present in an experimental or diagnostic sample containing multiple unique nucleic acids (Southern, Trends in Genetics 12:110-115 (1996)); hybridization assays which are able to identify genes which have a mutation such that the gene present in the experimental or diagnostic sample differs from that of the wild-type gene (Southern, WO 89/10977 (1989)); and in polymerase extension assays where the immobilized nucleic acids serve as primers for DNA synthesis by a DNA polymerase enzyme following hybridization to complementary target nucleic acids that may be present in the sample (Shumaker et al., Hum. Mut. 7:346-354 (1996); Syvanen et al., Am. J. Hum. Genet. 52:46-59 (1993)).

Presently, there are a number of known methods for covalently coupling a nucleic acid to a solid support for use in an experimental or diagnostic assay . These can be divided into two categories: 1) those in which preformed nucleic acids are coupled to the support; and 2) those in which the nucleic acids are synthesized in situ on the support.

In the first approach, the nucleic acids are deposited on the support either by hand or by automated liquid handling equipment (Lamture et al., Nucleic Acids Research 22:2121-2125 (1994); Yershov et al., Proc. Natl. Acad. Sci. USA 93:4913-4918 (1996)). To accomplish covalent attachment of the nucleic acids to the support, either the support, the nucleic acids, or both, are chemically activated prior to deposition. Alternatively, the nucleic acids can be deposited on the support and nonspecifically immobilized by physical means such as heat or irradiation with ultraviolet light (Life Science Research Product Catalog, BioRad Laboratories, Richmond, Calif., pg.269-273 (1996); Meinkoth and Wahl, Analytical Biochemistry 138:267-284 (1984)). In general, chemically mediated coupling is preferred since specific, well-defined attachments can be accomplished, thereby minimizing the risk of unwanted artifacts from the immobilization process.

In the second approach, oligonucleotides are synthesized directly on the support using chemical methods based on those used for solid phase nucleic acid synthesis (Southern et al., Nucleic Acids Research 22:1368-1373 (1994)). Recently, specialized apparatus and photolithographic methods have been introduced which allow the synthesis of many different oligonucleotides at discrete, well-defined positions on planar glass or silica supports (Pease et al., Proc. Natl Acad. Sci. USA 91:5022-5026 (1994)). In general, these methods are most useful for applications which require many hundreds or thousands or tens of thousands of different immobilized nucleic acids, such as for sequencing by hybridization, SNP analysis, or gene expression analysis.

Yet another method presently in use to couple a nucleic acid molecule to a solid support involves the formation of an electroconducting conjugated polymerized layer (Livache et al., Nucleic Acids Research 22:2915-2921 (1994)). This polymerized layer is formed by copolymerization of a mixture containing pyrrole monomers and oligonucleotides covalently linked to a pyrrole monomer. The copolymerization reaction initiates following application of an electrical charge through the electrode which has been placed into the mixture containing the copolymerizable components. The dimensions of the polymerized layer which coats the surface of the electrode can be varied by adjusting the surface area of the electrode which is placed into the mixture.

Each of the methods disclosed above have specific limitations. For instance, the polymerized layer which coats the surface of an electrode cannot be formed on a solid support which is not able to transmit an electrical charge into the mixture containing the copolymerizable monomer units. Most of the other disclosed methods are also limited to solid supports of a particular type. In addition, several of these methods require special types of equipment, and involve a degree of technical difficulty which may make it difficult to covalently link a nucleic acid molecule to a solid support in a reproducible manner.

Additional different attachment methods are also available for attaching synthetic capture probes or other nucleic acid sequences to solid surfaces. These include but are not limited to the following and the methods described in the following table:

Non-covalent methods, in which a capture probe is attached to a surface by interactions other than covalent chemical bonds. Examples include using biotinylated oligos bound to a surface functionalized with streptavidin (Gilles and Holmstrom), electrostatic adhesion of oligonucleotides to polystyrene or glass surfaces (Nikiforov) or polylysine functionalized surfaces (Shalon, Brown, and Running), non-covalent interaction of oligo with casein coated slides (Stimpson), and non-covalent interaction of specific ligand-receptor systems (Rogers, J. T.).

Covalent attachment methods, in which a covalent bond is formed between the capture probe and some functionality on the solid surface, include but are not limited to the examples in the following table:

| | | Nature | |
|---|---|---|---|
| Oligonucleotide | Surface | of covalent | |
| functionality | functionality | bond formed | Ref |
| Primary amine, | Aldehyde | Schiff's base; | Timofeev |
| either 3', 5', or | | secondary amine | |
| internal | | after reduction | |
| | | with BH4 | |
| 3'-OH or 5'-OH | Epoxy or thiol | Thioether | Shi |
| | modified glass | | |
| | surface | | |
| Aldehyde | Primary amine | Schiff's base; | Timofeev |
| | | secondary amine | |
| | | after reduction | |
| | | with BH4 | |
| di-aldehyde | hydrazide | Hydrazone | Yershov |
| (oxidized 3' | | | |
| terminal | | | |
| ribonucleotide) | | | |
| Primary amine | Activated acid | Amide | Zhang |
| Primary amine | epoxide | Secondary amine | Eggers |
| Primary amine | NHS Ester | Amide | Surmodics |
| Thiol | Bromoacetyl | Thio-ether | Fahy |
| Thio-phosphate | Bromoacetyl | Thio-phosphate | Gryaznov |
| Thiol | Gold | Gold-Sulfur | Beebe |
| | | complex | |
| Thiol | maleimide | Thioether | Chrisey |
| Activated acid | Primary amine | Amide | Joos |
| Maleimide | thiol | Thioether | Chrisey |
| Primary amine | phosphoramidite | Phosphoramidate | Schepinov |
| Primary amine | Activated | Isothiocyanate | Guo |
| | isothiocyanate | | |
| Thiol | thiol | Disulfide | Rogers, Y-H, |
| | | | and Anderson |

Chrisey, L. A., etal, (1996), NAR, v24,#15, p3031.

Timofeev, E. N., etal, (1996), NAR, v24, #16, p3142.

Schepinov, M. S., etal, (1997), NAR, v25, #6, p1155.

Nikiforov, T. T., etal, (1995), Anal. Biochem., 227, p201, and U.S. Pat. No. 5,610,287

Eggers, M., etal, (1994), Biotechniques, v17, #3, p516.

Guo, Z., etal, (1994), NAR, v22, #24, p5456.

Yershov, G., (1996), PNAS, v93, p4913.

Shalon, D. (1996), Genome Research, v6, p639.

Gilles, P. etal., (1999), Nat. Biotech., v17, p365.

Fahy, E., (1993) NAR, v21, #8, p1819.

Rogers, J. T., (1997), Gene Therapy, v4, p1387.

Rogers, Y-H., (1999), Anal. Biochem., v266, p23.

Zhang, Y. etal., (1991), NAR, v19, #14, p3929.

Joos, B., etal., (1997), Anal. Biochem., v247,p96.

Beebe, T. P., et al, (December 1995), U.S. Pat. No. 5,472,881

Shi, J., et al, (July 1999), U.S. Pat. No. 5,919,626

Anderson, et al, (November 1998), U.S. Pat. No. 5,837,860

Brown, P., et al, (September 1998), U.S. Pat. No. 5,807,522

Stimpson, D. I. et al, (1995), PNAS, v92, p6379

Holmstrom, K. et al, (1993), Anal. Biochem, v209, p278

Running, J. A, et al, (1990), Biotechniques, v8, p276

Other attachment methods may also be used that do not eliminate the ability of the probe or other nucleic acid molecule to hybridize with complementary sequences.

F. Binding Detection

A variety of arrangements can be used to detect the scattered light signal. For example, detection can be carried out as described in the Yguerabide et al. applications, supra. For a scattered light signal, the illumination source and the detector or detectors are configured to reduce background signal so that a sensitive assay results.

In general, for an array, the light scattering signal for each assay spot on the array is read. Reading can, for example, be performed as described in Schena, supra, with appropriate arrangement of illumination and detection.

### EXAMPLES

In general, an exemplary method of the invention detects specific mutations to identify the phenotypic classification of an individual from whom a sample was obtained. The user obtains blood or other biological sample, isolates genomic DNA using standard methodologies and subjects the genomic DNA to Polymerase Chain Reaction (PCR) amplification in the presence of biotinylated dUTP using primers specific for CYP2D6. The multiplex PCR reaction may generate two or more separate amplicons. The amplicons are denatured and hybridized to an array of capture oligonucleotides on a glass slide. Each of the capture oligonucleotides occupies a distinct location in the array and is specific for either a mutant or wildtype CYP2D6 allele. RLS particles coated with antibody to biotin are used to detect hybridized biotinylated PCR amplicons, and a signal is obtained from the RLS Particles by measuring light scattering. Results are analyzed and if mutations corresponding to a specific allele of the CYP2D6 gene are detected, the allele is specified using standard nomenclature.

### Example 1

### CYP2D6 Allele Selection for Exemplary Assay

As indicated above, the exemplary assay described utilizes 5 different alleles with their associated polymorphic sites and respective mutant sequences. The alleles are indicated in the following table with the phenotypic characterization and prevalence.

Alleles for Current Assay

| | | Enzyme Activity/ | |
|---|---|---|---|
| Allele (Number) | Allele (Letter) | Phenotype | Prevalence¹ |
| CYP2D6*1 | WT | Extensive | ˜85% |
| | | Metabolizer | |
| CYP2D6*3 | A | Poor Metabolizer | ˜2% |
| CYP2D6*4 | B | Poor Metabolizer | ˜9% |
| CYP2D6*6 | T | Poor Metabolizer | ˜0.9% |
| CYP2D6*7 | B | Poor Metabolizer | ˜0.5% |
| CYP2D6*9 | C | Intermediate to Poor | ˜1.5% |
| | | Metabolizer | |

¹Prevalence is indicated as a percent in Caucasian populations unless otherwise indicated.

Slide Preparation

Slides can be prepared as previously described using coated slides and attaching appropriate CYP2D6 capture probes.

### Example 2

### Probe and primer preparation

Exemplary CYP2D6 primers:

| | |
|---|---|
| Name | Primer sequence |

| | | |
|---|---|---|
| CYPwt(−)3049 | 5′-CTCGGCCCCTGCACTGTTTC-3′ | SEQ ID NO.1 |
| CYPwt(−)1951 | 5′-GCTTTGTCCAAGAGACCGTTG- | SEQ ID NO.2 |
| | 3′ | |
| CYPwt(+)2165 | 5′-CTCGGAAGAGCAGGATTTGCGT | SEQ ID NO.3 |
| | A-3′ | |
| CYPwt(+)2603 | 5′-CCTGACCCAGCTGGATGAG-3′ | SEQ ID NO.4 |
| CYPwt(+)1473 | 5′-CTTCCCTGAGTGCAAAGGCG- | SEQ ID NO.5 |
| | 3′ | |

In the present exemplary embodiment, two sets or a total of four primers are used in a multiplex PCR reaction. 1473(+) and 1951(−) generate a 499 bp amplicon harboring the B, G and T alleles. 2165(+) or 2603(+) and 3049(−) generate to second amplicon that is either 904 bp for 2165(+) or 466 bp for 2603(+) harbors the A, C and E alleles. The locations of the primers are show schematically in figure FIND FIGURE representing the relative locations of the system's probes and primers.

With respect to primer nomenclature: CYP refers to CYP2D6, wt refers to wildtype sequence, (+) or (−) refers to the sense of the primer relative to the coding (+) sense strand, number in each primer name reflects the 5′ nucleotide base corresponding to the reference CYP2D6 gene sequence submitted to Genbank by F. J. Gonzalez, CYP2D6 (denoted originally as CYP2DG), Genbank accession number M33189.1).

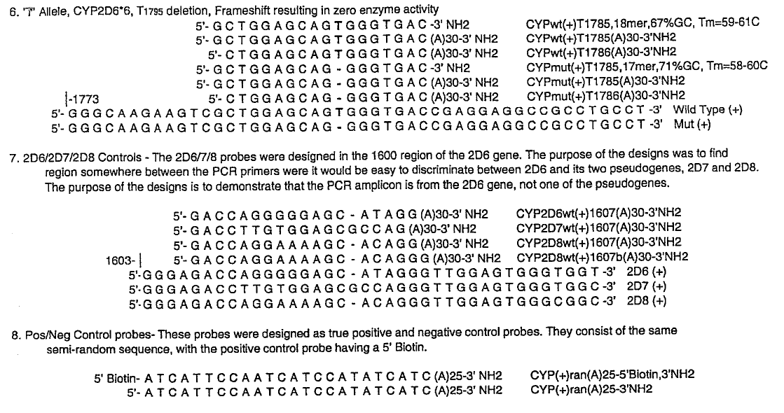

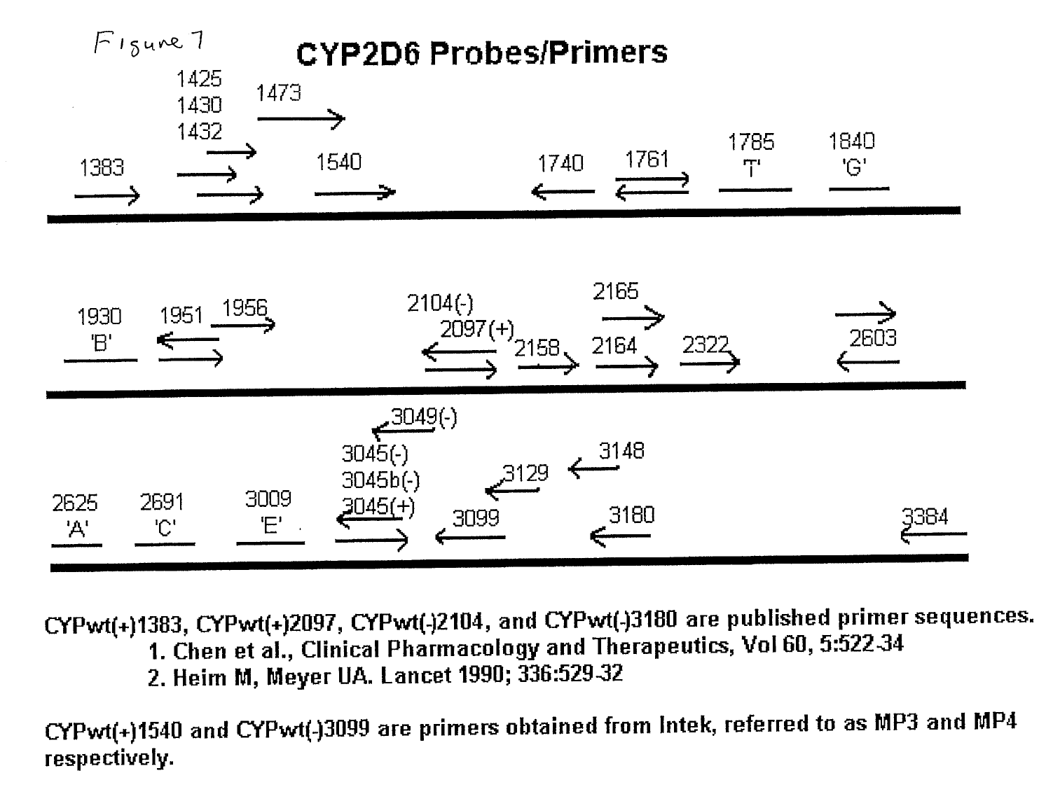

Probes are utilized as shown in FIG. 6 FIND FIGURE for detection of immobilized target CYP2D6 nucleic acid molecules.

In general, probes and primers are prepared by the usual synthetic methods. Alternatively, enzymatic synthesis could also be utilized.

Specimen Preparation: Isolation of Genomic DNA

Isolate genomic DNA from patient blood using any of a variety of methods, e.g., using a qualified commercial kit. The purified genomic DNA is suspended or eluted in a low ionic strength Tris-based buffer (e.g. 4 mM Tris, 0.01 mM EDTA pH 8.3) and stored at 2° to 8° C. The quality and concentration of the DNA is evaluated by measuring an absorbance ratio at 260/280 nm (A_{260/280}) on an aliquot of the prepared genomic DNA. Acceptable preparations of genomic DNA will yield A_{260/280} values generally from 1.8 to 2.0.

PCR

PCR can be performed on the prepared genomic DNA sample with many variations. In this example, the PCR amplification was performed in the following manner. An aliquot of the sample was diluted in a low ionic strength buffer to a working concentration of approximately 5 ng/μL.

A PCR mixture is made as follows: (Per reaction)

| Component | Volume | |
|---|---|---|
| Water | 21.6 | μL |
| 5X PCR Buffer | 10.0 | μL |
| Biotin₁₁-dUTP (1 mM stock) | 3.0 | μL |
| MDS-CYP2D6 Primer Mix | 5.0 | μL |
| Taq polymerase | 0.4 | μL |
| (PE AmpliTaq ™ , 5 U/mL stock) | | |
| gDNA (5 ng/μL stock) | 5.0 | μL |
| Total Volume | 45.0 | μL |

PCR is conducted using the following program:

| Cycles/Step | | | Temperature | Time | |
|---|---|---|---|---|---|
| 1 | cycle | denature | 95° C. | 5 | minutes |

After 1 minute at 95° C. add 5 μL of dNTP mix (10 mM stock)
directly into PCR mixture

| | | | | | |
|---|---|---|---|---|---|
| 30 | cycles | denature | 95° C. | 45 | seconds |
| | anneal | 64° C. | 30 | seconds | |
| | extend | 72° C. | 1 | minute | |
| 1 | cycle | extend | 72° C. | 10 | minutes |

Assay Setup

Pre-heat a circulating waterbath to 50° C.

Pre-heat a heat-block or thermocycler to 95° C.

Insert the nozzle into the plastic squeeze bottle containing wash buffer.

Reagent Preparation

Sufficient RLS particles are preferably provided for a binding reaction to fully bind to all immobilized labeled target molecules. The appropriate amount can be readily determined by empirically optimizing detection results. Typically, RLS particles are diluted to a final concentration between 1.0 and 3.0 optical density units prior to binding to the captured target sequence.

Amplicon Preparation

After reaction, the PCR amplicon products are denatured by heating and then cooled to an appropriate temperature for hybridization in a buffer suitable for hybridization. Typically, 25 μL of the PCR reaction is transferred to a new microfuge tube. 25 μL hybridization buffer is added to prepared amplicon in a microfuge tube followed by denaturation of the prepared amplicon mixture for 10 minutes at 95° C. This sample is cooled in a microfuge tube in water bath at room temperature (20° to 30° C.) for 1 minute.

Hybridization

Assay slides are placed in a hydration chamber to provide a controlled environment with high relative humidity to control evaporation of the hybridization solution. 25 μL of hybridization mixture is transferred from microfuge tube to slide/chip ensuring that the designated reaction area is completely covered by the hybridization mixture. Slides are then incubated for 30 minutes at room temperature (20° to 30° C.). During the 30 minute incubation, the temperature of wash buffer is equilibrated. For each sample to be tested, approximately 15 mL of the buffer is placed in an reaction vessel (each vessel holds 4 slides). Once slides are loaded, the reaction vessel lid is tightly sealed and the reaction vessel is placed in a floating rack and placed in a 50° C. circulating water bath.

After hybridization, slides are removed from the hydration chamber and washed thoroughly with wash buffer.

SNP Discrimination

Slides are then immediately placed in preheated reaction vessels in the 50° C. circulating water bath and incubated for approximately 10 minutes.

The slides are removed from the reaction vessel and placed in the hydration chamber.25 μL blocking solution Block is added to the slide. Following an incubation for 10 minutes at room temperature (20° to 30° C.), the slides are removed from the hydration chamber and washed with wash buffer.

RLS Signal Generation

25 μL of prepared 1X RLS Particles are added to the slide ensuring that the designated reaction area is completely covered by the RLS Particles.In the hydration chamber, the slides are incubated for 10 minutes at room temperature (20° to 30° C.).

Following RLS particle binding, the slides are removed from the hydration chamber and washed with wash buffer.

RLS Signal Detection

Excess wash buffer is drained from the slide surface and the underside of the slide is dried.

For imaging, two drops of microscope immersion oil are placed on the viewing prism directly beneath the objective lens and the slide is placed on the viewing prism on the RLS detection instrument with the array facing upwards.

The spots representing the specific CYP2D6 alleles are visualized and the image pattern is recorded using an image analysis and genotype calling software program.

### RESULTS

Preferably an assay specific computer program is used for collecting and analyzing the assay results. The software will locate and analyze the intensity of individual hybridization spots and will display an analysis of the positive controls, negative controls and signal controls.

Reports: The results of the test can be reported in various ways for example: (1) as actual images of the RLS signals on the slide, (2) as a table specifying the finctionality of the control spots and (3) as the actual “call” of the sample genotype. Reports can also provide sample information input by the user and will indicate if any aspect of the test is not within assay specifications or if the results are out of range.

Target Control Profile and Slide Spotting

| | | Proposed | |
|---|---|---|---|
| | | Slide | |
| Control | Number | Location | Function |
| External | 1 | C2,R1 | An oligonucleotide sequence specific |
| Positive | | | for CYP2D6. This control ensures the |
| Control | | | function and specificity of the assay in |
| | | | its entirety. Also see, Internal |
| | | | Positive Control |
| External | 1 | C2,R2 | A random oligonucleotide sequence |
| Negative | | | with no homology to the CYP2D6 gene. |
| Control | | | This control ensures that the CYP2D6 |
| | | | PCR amplicon does not hybridize non- |
| | | | specifically. Also see, Negative Signal |
| | | | Control |
| Internal | 1 | C2,R1 | An oligonucleotide sequence specific |
| Positive | | | for CYP2D6. This control ensures the |
| Control | | | PCR reaction has yielded sufficient |
| | | | CYP2D6 amplicon for detection using |
| | | | the RLS particles. Also see, External |
| | | | Positive Control |
| Internal | 1 | C2,R3 | An oligonucleotide sequence specific |
| Negative | | | for the CYP2D7 gene. CYP2D7 is a |
| Control | | | non-expressed pseudogene of CYP2D6. |
| #1 | | | This control ensures that non-specific |
| | | | CYP2D7 amplicons have not been |
| | | | generated during the PCR procedure. |
| Internal | 1 | C2,R4 | An oligonucleotide sequence specific |
| Negative | | | for the CYP2D8 gene. CYP2D8 is a |
| Control | | | non-expressed pseudogene of CYP2D6. |
| #2 | | | This control ensures that non-specific |
| | | | CYP2D8 amplicons have not been |
| | | | generated during the PCR procedure. |
| Positive | 6 | C1,R1 | Control spots containing the bio- |
| RLS | | C1,R5 | tinylated form of the External Negative |
| Signal | | C2,R1 | Control oligonucleotide. These spots |
| Control, | | C2,R5 | ensure the integrity of the RLS particles |
| Positional | | C7,R5 | both to bind and to yield a signal. And |
| Control | | | are used by the imaging software for |
| | | | alignment. Also see, RLS Positive |
| | | | Control Signals. |
| Negative | 1 | C1,R2 | Control spots containing non-bio- |
| RLS | | | tinylated versions of the targets |
| Signal | | | present in the RLS positive control |
| Control | | | spots. Also see, External Negative |
| | | | Control. |

Exemplary Spot Layout

| | | | | |
|---|---|---|---|---|
| Signal | External/ | Wild-Type | Mutant | |
| Generation, | Internal | CYP2D6*3 | CYP2D6*3 | |
| Positional | Positive | | | |
| Control | Control | | | |
| | 2D6 | | | |
| Negative | External | Wild-Type | Mutant | |
| Signal | Negative | CYP2D6*4 | CYP2D6*4 | |
| Generation | Control | | | |
| | XX | | | |
| | Internal | Wild-Type | Mutant | |
| | Negative | CYP2D6*6 | CYP2D6*6 | |
| | Control | | | |
| | 2D7 | | | |
| | Internal | Wild-Type | Mutant | |
| | Negative | CYP2D6*7 | CYP2D6*7 | |
| | Control | | | |
| | 2D8 | | | |
| Signal | Signal | Wild-Type | Mutant | Signal |
| Generation, | Generation, | CYP2D6*8 | CYP2D6*8 | Generation, |
| Positional | Positional | | | Positional |
| Control | Control | | | Control |

### QUALITY CONTROL

Assay Controls: The test slide contains 8 control oligonucleotides:

External Positive Control: An oligonucleotide sequence specific for CYP2D6. This control ensures the function and specificity of the assay in its entirety. Also see, Internal Positive Control

External Negative Control: A random oligonucleotide sequence with no homology to the CYP2D6 gene. This control ensures that the CYP2D6 PCR amplicon does not hybridize non-specifically. Also see, Negative Signal Control

Internal Positive Control: An oligonucleotide sequence specific for CYP2D6. This control ensures the PCR reaction has yielded sufficient CyP2D6 amplicon for detection using the RLS particles. Also see, External Positive Control

Internal Negative Control 1: An oligonucleotide sequence specific for the CYP2D7 gene. CYP2D7 is a non-expressed pseudogene of CYP2D6. This control ensures that non-specific CYP2D7 amplicons have not been generated during the PCR procedure.

Internal Negative Control 2: An oligonucleotide sequence specific for the CYP2D 8 gene. CYP2D8 is a non-expressed pseudogene of CYP2D6. This control ensures that non-specific CYP2D8 amplicons have not been generated during the PCR procedure.

RLS Positive Control Signals: Control spots containing the biotinylated form of the External Negative Control oligonucleotide. These spots ensure the integrity of the RLS particles both to bind and to yield a signal. Present at several sites within the array. Also see, Positional Control Spots.

RLS Negative Signal Control: Control spots containing non-biotinylated versions of the targets present in the RLS positive control spots. Also see, External Negative Control.

Positional Control Spots: Control spots present at several sites within the array for use by the imaging software for alignment. Also see, RLS Positive Control Signals.

### REFERENCES

Phillips, IR. and Shephard, EA (eds), “Cytochrome P450 Protocols”, Volume 107, Humana Press, 1998.

Richardson, JH. and W. Emmett Barkley (eds), “Biosafety in Microbiological and Biomedical Laboratories.” HHS Publication Number [CDC]88-8395.

Steiner, E, Bertilsson L, Sawe J, Bertling I, Sjoqvist F. “Polymorphic Debrisoquin Hydroxylation in 757 Swedish Subjects.” Clin Pharmacol Ther 1998; 44:431-5

Daly AK, Brockmiller J., Broly F., et al. “Nomenclature for Human CYP2D6 Alleles.” Pharmacogenetics 1996;6:193-201

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. For example, using other light scattering particles, and/or methods probe binding are all within the scope of the present invention. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values are provided for embodiments, additional embodiments are described by taking any 2 different values as the endpoints of a range.

Thus, additional embodiments are within the scope of the invention and within the following claims.

What is claimed is:

1. A method for determining the presence or absence of a CYP2D6 target sequence in a sample of DNA containing nucleic acid corresponding to CYP2D6, comprising
contacting said nucleic acid with a probe under stringent binding conditions, and detecting the presence or absence of target sequence bound with said probe, wherein said target sequence or said probe is bound with scattered light detectable particle, and said detecting comprises observing light scattered from said particle as an indication of said presence or absence.

2. The method of claim 1, further comprising amplifying a portion of said nucleic acid corresponding to CYP2D6, and contacting the amplified nucleic acid with said probe.

3. The method of claim 1, wherein a plurality of capture probes comprising nucleotide sequence complementary to nucleic acid corresponding to CYP2D6 are immobilized on a solid surface.

4. The method of claim 1, wherein said determining the presence or absence of at least one target sequence in said nucleic acid corresponding to CYP2D6 comprises determining the presence or absence of a plurality of target sequences using a plurality of different probes.

5. The method of claim 4, wherein the presence or absence of said plurality of target sequences identifies at least one CYP2D6 allele.

6. The method of claim 4, wherein a plurality of different nucleic acid molecules corresponding to CYP2D6 is immobilized at different spots on a solid surface.

7. The method of claim 1, further comprising demonstrating that nucleic acid sequence from CYP27D or CYP2D8 pseudogenes or both is not amplified.

8. The method of claim 5, wherein said at least one allele comprises a plurality of alleles.

9. The method of claim 1, wherein said target sequence is labeled by incorporation labeling.

10. An amplification oligonucleotide primer adapted for amplifying a portion of a CYP2D6 gene comprising a sequence polymorphism, wherein said oligonucleotide binds to an intron of said CYP2D6 gene.

11. The primer of claim 10, wherein said primer is a gene-specific primer.

12. The amplification oligonucleotide primer of claim 11, wherein said primer hybridizes under stringent hybridization conditions to a CYP2D6 target site, wherein said primer contains at least one nucleotide at the 3′ end that base pairs with a complementary nucleotide in a CYP2D6 target sequence in at least one allele and does not base pair with a complementary nucleotide in a CYP2D6 target sequence in at least one different allele.

13. The primer of claim 11, wherein said primer is an allele-specific primer.

14. The amplification oligonucleotide primer of claim 11, wherein said oligonucleotide comprises a sequence selected from the group consisting of
5′-CTCGGCCCCTGCACTGTTTC-3′,
5′-GCTTTGTCCAAGAGACCGTTG-3′,
5′-CTCGGAAGAGCAGGATTTGCGTA-3′,
5′-CCTGACCCAGCTGGATGAG-3′, and
5′-CTTCCCTGAGTGCAAAGGCG-3′.

15. An amplification oligonucleotide primer adapted for amplifying a portion of a CYP2D6 gene comprising a sequence polymorphism, wherein said primer is a gene-specific primer.

16. An allele specific probe, comprising a molecule that preferentially binds to a labeled CYP2D6 target nucleic acid sequence at least partially comprising a sequence corresponding to a polymorphism in a CYP2D6 gene, wherein a scattered light detectable particle 1-500 nm in size is bound with said probe.

17. The probe of claim 16, wherein said probe comprises a nucleotide sequence that is at least 80% complementary to said CYP2D6 target nucleic acid sequence.

18. The probe of claim 20, wherein said probe comprises a nucleotide sequence that is designed to discriminate different allelic forms of at least one CYP2D6 target nucleic acid sequence.

19. The probe of claim 16, further comprising a spacer region.

20. The probe of claim 19 wherein said spacer region comprises a polynucleotide tail.

21. The probe of claim 20, wherein said polynucleotide tail is 10-50 nucleotides in length.

22. A labeled target nucleic acid molecule corresponding to a polymorphic site-containing portion of CYP2D6, wherein said label provides binding of a light scattering particle.

23. The molecule of claim 22, wherein said label is a hapten.

24. The molecule of claim 23, wherein a specific hapten-binding molecule bound with a light scattering particle is bound to said hapten.

25. The molecule of claim 22, wherein said label is a modified nucleotide.

26. The molecule of claim 25, wherein an antibody recognizing said modified nucleotide is bound to said molecule and said antibody is bound with a light scattering particle.

27. An isolated CYP2D6 nucleic acid sequence, wherein said nucleic acid sequence is bound with a probe and said nucleic acid sequence or said probe is bound with a scattered light detectable particle.

28. The nucleic acid sequence of claim 27, wherein said probe is a detection probe with a scattered light detectable particle bound to said probe.

29. The nucleic acid sequence of claim 27, wherein said probe is a capture probe.

30. The nucleic acid molecule of claim 27, wherein said probe is an allele-specific probe.

31. The nucleic acid sequence of claim 27, wherein said scattered light detectable particle includes or is bound to a first member of a binding pair, said first member of a binding pair is bound with the second member of the binding pair; and said second member of the binding pair is bound with said probe or said nucleic acid sequence.

32. The nucleic acid sequence of claim 27, wherein said sequence comprises an incorporated label.

33. The nucleic acid sequence of claim 32, wherein said label is a hapten.

34. The nucleic acid sequence of claim 32, wherein said label is a modified nucleotide recognized by an antibody.

35. A method for determining the presence of a CYP2D6 allele in a nucleic acid sample that may contain nucleic acid corresponding to CYP2D6, comprising
contacting said nucleic acid sample with at least one allele-specific probe under conditions wherein said at least one probe specifically binds to any said nucleic acid corresponding to CYP2D6 in said sample that includes a specific sequence polymorphism and not to nucleic acid corresponding to CYP2D6 that does not include said specific sequence polymorphism,
binding said nucleic acid corresponding to CYP2D6 that includes said specific sequence polymorphism or said probe with a scattered-light detectable particle of a size between 1 and 500 nm inclusive;
illuminating any said particles associated with probe bound with said nucleic acid corresponding to CYP2D6 with light under conditions which produce scattered light from said particles and in which light scattered from one or more said particles can be detected; and
detecting said light scattered by any said particles under said conditions as a measure of the presence of said nucleic acid corresponding to CYP2D6 including said specific sequence polymorphism.

36. The method of claim 35, where said illuminating is with non-evanescent wave, and said scattered light can be detected by a human eye with less than 500 times magnification and without electronic amplification.

37. The method of claim 35, wherein said probe comprises a nucleic acid sequence that hybridizes with said nucleic acid corresponding to CYP2D6.

38. The method of claim 35, further comprising labeling nucleic acid corresponding to CYP2D6 by incorporation labeling.

39. A kit adapted for determination of the presence of at least one CYP2D6 sequence polymorphism in CYPD6 nucleic acid, comprising
at least one solid phase array, wherein said array chip is adapted to bind CYP2D6 nucleic acid at a plurality of spots;
at least one allele specific probe that specifically binds to a CYP2D6 target sequence; and
at least one distinguishable type of scattered light detectable particle 1 to 500 nm in size that binds to said CYP2D6 nucleic acid.

40. The kit of claim 39, wherein said solid phase array is adapted to bind a plurality of different CYP2D6 nucleic acid molecules at different spots.

41. The kit of claim 39, wherein said at least one allele-specific probe comprises a plurality of different allele-specific probes.

42. The kit of claim 39, wherein said allele-specific probe is a capture probe bound on said solid phase array.

43. The kit of claim 39, further comprising one or more components for incorporation labeling of target nucleic acid.

44. The kit of claim 43, wherein said components comprise at least one component selected from the group consisting of a DNA polymerase, a modified nucleotide comprising a hapten, and a modified nucleotide comprising a modified nucleotide recognized by an antibody.

45. The kit of claim 39, wherein said at least one distinguishable type is a plurality of distinguishable types.

46. A kit adapted for determination of the presence of at least one CYP2D6 sequence polymorphism in CYP2D6 target nucleic acid, comprising
at least one allele-specific probe that specifically binds to a CYP2D6 target sequence; and
at least one distinguishable type scattered light detectable particle adapted to bind with said allele-specific probe or with said target sequence.

47. The kit of claim 46, further comprising at least one CYP2D6 solid phase array.

48. The kit of claim 46, wherein said at least one allele-specific probe comprises a plurality of different allele-specific probes.

49. The kit of claim 46, wherein said at least one type scattered light detectable particle is bound to said at least one allele-specific probe.

50. The kit of claim 46, wherein said at least one type scattered light detectable particle is a plurality of different type particles.

51. The kit of claim 46, further comprising at least one amplification oligonucleotide primer adapted to bind to or extend through a CYP2D6 polymorphic site.

52. The kit of claim 51, wherein said at least one amplification oligonucleotide primers comprises a plurality of said amplification oligonucleotide primers.

53. The kit of claim 46, further comprising at least one component adapted for incorporation labeling of target nucleic acid molecules.

54. The kit of claim 53, wherein said at least one component includes a DNA polymerase suitable for non-PCR enzymatic synthesis of target molecules.

55. The kit of claim 53, wherein said at least one component includes a hapten-linked nucleoside triphosphate or a modified nucleotide recognized by an antibody.

56. An oligonucleotide comprising
a nucleotide sequence and an incorporated label,
wherein the sequence of said oligonucleotide corresponds to a portion of a gene, said portion comprising a polymorphic site.

57. The oligonucleotide of claim 56, wherein said oligonucleotide includes a hapten.

58. The oligonucleotide of claim 56, wherein said oligonucleotide includes a modified nucleotide that will specifically bind an antibody.
